(19) United States
(12) Patent Application Publication
(10) Pub. No.: US 2013/0202670 A1
(43) Pub. Date: Aug. 8, 2013

(54) BIOACTIVE ANTIBACTERIAL BONE GRAFT MATERIALS CONTAINING SILVER

(71) Applicant: ORTHOVITA, INC., Malvern, PA (US)

(72) Inventors: Marissa M. Darmoc, Philadelphia, PA (US); Theodore D. Clineff, Phoenixville, PA (US); Lauren S. Brown, Media, PA (US); Kristi L. Wagner, Coatesville, PA (US)

(73) Assignee: ORTHOVITA, INC., Malvern, PA (US)

(21) Appl. No.: 13/747,933

(22) Filed: Jan. 23, 2013

Related U.S. Application Data
(60) Provisional application No. 61/594,805, filed on Feb. 3, 2012.

(51) Int. Cl. A61L 27/40

(52) U.S. Cl. CPC A61L 27/40
USPC 424/405; 424/618

(57) ABSTRACT

The present invention generally relates to silver-containing bioactive antibacterial materials and composites that enhance bone growth while preventing surgical site infection. The present invention also relates to bioactive antibacterial materials and composites that include a bimodal bioactive glass particle size distribution. The bioactive antibacterial composite finds utility in a variety of clinical applications including spine and orthopaedic procedures.

The present application claims the benefit of the filing date of U.S. Provisional Patent Application No. 61/594,805 filed Feb. 3, 2012, the disclosure of which is hereby incorporated herein by reference.

### BACKGROUND OF THE INVENTION

Surgical site infections (SSI) present a significant clinical problem in both spine and orthopaedic surgery. As well as being costly to the health care system, these infections interfere with wound healing and therefore prolong recovery time for patients. There is a need in the field for treatments that prevent surgical site infection at the local delivery site. The present invention addresses this need by providing a composite biomaterial that includes at least one antibacterial component for preventing surgical site infections.

Biomaterials, including various metals, polymers and ceramics, have been used as implant materials in the field of spine, orthopaedics and dentistry including fusion, trauma, fracture repair, reconstructive surgery and alveolar ridge reconstruction, for over a century due to their biocompatibility and physical properties. Among these biomaterials, porous calcium phosphate-based bone grafts are known in the art for use in filling bony voids or gaps in the skeletal system. Examples of such bone grafts are described in, for example, U.S. Pat. No. 6,991,803; U.S. Pat. No. 6,521,246; and U.S. Pat. No. 6,383,519, incorporated herein. Vitoss® Bone Graft Substitute (Orthovita, Inc., Malvern Pa.) is one exemplary type of such bone grafts. Such porous calcium phosphate-based bone grafts have been further modified and improved to incorporate biocompatible materials such as, for example, polymers including collagen, to impart improved handling ability of the bone graft; and bioactive glasses, to further enhance the biological activity of the bone graft. Examples of such materials are described in, for example, U.S. Pat. No. 7,534,451; U.S. Pat. No. 7,531,004; U.S. Pat. No. 7,189,263 and U.S. Patent App. No. 20080187571, incorporated herein. Vitoss® BA Bioactive Bone Graft Substitute (Orthovita, Inc., Malvern, Pa.) is one exemplary type of such a bone graft incorporating bioactive glass.

Bioactive (BA) glasses have been extensively studied for their bone bonding properties. The use of BA glass alone and in combination with other materials is generally described in U.S. Pat. No. 5,681,872; U.S. Pat. No. 5,914,356; and U.S. Pat. No. 6,987,136, each of which is assigned to the assignee of the present invention and is incorporated in this document by reference in its entirety.

The wound healing and bactericidal properties of BA glasses have also been reported, particularly BA glasses of certain particle size ranges. U.S. Pat. No. 6,756,060; No. 6,428,800 and U.S. Pat. No. 5,834,008 describe wound and burn dressings comprising BA glass. However, the BA glass is generally combined with topical antibiotic and incorporated into bandages.

Accordingly, there is a need in the art for implant materials and composites that induce bone formation and prevent surgical site infections. There is also a need in the art for a method of preparing a homogeneous bioactive antibacterial composite; and for methods of using bioactive antibacterial materials and composites in a variety of clinical applications including spine and orthopaedic procedures. The present invention fulfills these needs.

### BRIEF DESCRIPTION OF SEVERAL VIEWS OF THE DRAWINGS

The invention is best understood from the following detailed description when read in connection with the accompanying figures. It is emphasized that, according to common practice, the various features of the figures are not to scale. On the contrary, the dimensions of the various features are arbitrarily expanded or reduced for clarity. Included are the following figures:

FIG. 1 is a graphical depiction of the in-vivo release and degradation profile of the bioactive antibacterial material of the present invention.

FIG. 2A illustrates one basic form of the bioactive antibacterial graft material in cylinder form.

FIG. 2B depicts the graft material in cylindrical form 80 inserted into a bone void 83 below the femur 81 in the tibial plateau 82 within a human knee.

FIG. 3 illustrates another basic form of the present invention in strip form.

FIG. 4A illustrates one embodiment of the bioactive antibacterial graft material of the present invention in semi-spherical form used as a graft containment device. FIG. 4B depicts a semi-spherical form of the graft material 102 used to accommodate an artificial implant 103. The graft material 102 contains an acetabular cup 106, which holds a polyethylene cup 105, in this embodiment.

FIG. 5A illustrates the graft material of the present invention in disc form. FIG. 5B illustrates another embodiment of the bioactive antibacterial graft material of the present invention used as a cranio-maxillofacial 76, zygomatic reconstruction 72, and mandibular implant 74.

FIG. 6 illustrates one embodiment of a bioactive antibacterial graft material described shaped into a block/wedge form and used as a tibial plateau reconstruction that is screwed, bonded, cemented, pinned, anchored, or otherwise attached in place.

FIGS. 7A and 7B illustrate synthetic resorbable defect filling bone graft materials 272 for bone restoration having mesh 270 attached to one side. FIG. 7C depicts a synthetic resorbable defect filling bone graft material block in which the mesh 270 is sandwiched between the graft material 272.

FIGS. 8A, 8B, and 8C illustrate an embodiment of the bioactive antibacterial graft material of the present invention in semi-tubular form used as a long bone reinforcement sleeve. As shown in the figures, the semi-tube may have a moon cross-section with a uniform thickness (FIG. 8A); or a crescent moon cross-section with a tapered radius that comes to a point (FIG. 8B) or a tapered radius that is rounded on the edges (FIG. 8C).

FIGS. 9A, 9B and 9C are scanning electron microscope (SEM) images of one embodiment of the present invention in morsel form showing the bimodal particle size range of the glass (<53 μm and 90 μm-150 μm). FIG. 9A—×50 magnification (top), FIG. 9B—×250 magnification (middle), FIG. 9C ×500 magnification (bottom).

FIG. 10 is an SEM image (x200 magnification) of another embodiment of the present invention comprising beta-tricalcium phosphate and collagen in combination with the bimodal glass (i.e., Combeite glass-ceramic (<53 μm and 90 μm-150 μm).

FIG. 11 shows and exemplary particle size distribution graph of bimodal glass according to the present invention embodiment shown in FIGS. 9A, 9B and 9C.

FIG. 12 depicts an SEM image (×300 magnification) of the embodiment shown in FIG. 10 representing the bioactive nature of the material after submersion in simulated body fluid (SBF) for 1 day.

FIG. 13 depicts an SEM image (×300 magnification) of the embodiment shown in FIG. 10 representing the bioactive nature of the material after submersion in simulated body fluid (SBF) for 3 days.

FIG. 14 depicts an SEM image (×300 magnification) of the embodiment shown in FIG. 10 representing the bioactive nature of the material after submersion in simulated body fluid (SBF) for 7 days.

FIG. 15 depicts an SEM image (×300 magnification) of the embodiment shown in FIG. 10 representing the bioactive nature of the material after submersion in simulated body fluid (SBF) for 14 days.

FIG. 16 is a faxitron image of various embodiments of the present invention (in putty-like or “pack” form) comprising beta-tricalcium phosphate and collagen admixed with the bimodal glass showing the radiopacity of each in comparison to materials that do not contain the bimodal glass particle distribution.

FIG. 17 is a graphical depiction of the log reduction achieved at 1 day for increasing concentrations of bimodal glass incorporated into a bone graft substitute of collagen and calcium phosphate.

FIG. 18 depicts the results of an example experiment showing the log reduction of S. aureus in the presence of silver-containing bioactive (AgBA) glass at concentrations of 3, 5 and 7 mg/ml over 4 days.

FIG. 19 depicts the results of an experiment assessing alkaline phosphatase (ALP) secretion levels at increasing concentrations of AgBA at 21 days. ALP levels were normalized to protein content.

FIG. 20 depicts an SEM image of Saos-2 osteosarcoma cells at 21 days proliferating on a calcium phosphate scaffold which was cultured in 7 mg/mL AgBA glass extracted in MEM.

FIG. 21 depicts an SEM image of Saos-2 osteosarcoma cells at 21 days proliferating on a calcium phosphatescaffold which was cultured in 7 mg/mL AgBA glass extracted in MEM.

FIG. 22 depicts the results of an example experiment showing a substantial reduction of S. aureus colony forming units in the presence AgBA +PACK at 7, 11 and 15 mg/ml AgBA. The log concentrations measured at 0 hr, 24 hr, 5 day and 7 day were same for 11 mg/ml and 15 mg/ml AgBA. Also, the log concentrations measured at 5 day and 7 day for 7 mg/ml AgBA were same as 11 mg/ml and 15 mg/ml AgBA.

FIG. 23 depicts an SEM image of silver-containing Combeite glass-ceramic particles <53 μm in size at 1,500× magnification.

FIG. 24 depicts an SEM image of silver-containing Combeite glass-ceramic particles <53 μm in size at 2,500× magnification.

### DETAILED DESCRIPTION OF THE INVENTION

The present invention generally relates to bioactive antibacterial implant materials capable of preventing surgical site infection, and, more particularly to bioactive antibacterial materials that include bioactive glass and silver. In some embodiments, the bioactive glass is present in a specific bimodal glass size distribution. The present invention also relates to flexible and pliable bioactive antibacterial composites of silver, biocompatible polymer, ceramic and bioactive glass. In some embodiments, the bioactive glass is present in a specific bimodal glass size distribution. The present invention further relates to methods of repairing or fusing bone; and methods of facilitating bone repair while preventing surgical site infection.

The present invention provides silver-containing bioactive antibacterial materials and composites comprising bioactive and biocompatible implant materials for formulation of shaped bodies capable of inducing bone formation and preventing surgical site infection. The present invention also provides silver-containing bioactive composites that can be locally delivered and have the appropriate properties to prevent surgical site infection while stimulating bone formation. The present invention also provides for shaped bodies prepared from these materials and compositions to be used in a wide array of clinical applications including spinal and orthopaedic procedures.

As used herein, the term “antibacterial” includes both bactericidal activity and bacteriostatic activity and thus refers to a material that is capable of killing bacteria outright and also refers to a material that is able to stop additional growth of bacteria.

In one embodiment, the present invention comprises bioactive antibacterial materials, including silver-containing bioactive glass having particles with a particle size of less than about 53 microns (μm). In another embodiment, the present invention comprises bioactive antibacterial materials, including silver-containing bioactive glass having particles with a particle size ranging from less than about 53 μm to about 150 μm. In a further embodiment, the present invention comprises bioactive antibacterial materials, including silver-containing bioactive glass having particles with a particle size of less than about 53 μm and bioactive glass particles of a particle size ranging from about 90 μm to about 150 μm. In yet a further embodiment, the present invention comprises bioactive antibacterial materials, including silver-containing bioactive glass having particles with a particle size of less than about 90 μm and bioactive glass particles of a particle size ranging from about 90 μm to about 150 μm. In still a further embodiment, the present invention comprises bioactive antibacterial materials, including silver-containing bioactive glass having particles with a particle size ranging from about 32 μm to about 90 μm and bioactive glass particles of a particle size ranging from about 90 μm to about 150 μm.

In another embodiment, the present invention provides a bioactive antibacterial composite that includes silver, a biocompatible polymer and an inorganic ceramic in combination with bioactive glass. In some embodiments, the bioactive antibacterial composite of the invention that includes silver, a biocompatible polymer and an inorganic ceramic in combination with bioactive glass, has a bimodal glass size distribution. As used herein, the term “biocompatible polymer” refers to a polymer that, when introduced into a living system, will be compatible with living tissue or the living system (e.g., by not being substantially toxic, injurious, or not causing immunological rejection). In the present invention, the biocompatible polymer may be selected such that it will function to reinforce the composite in order to, for example, provide flexibility, pliability and structure to the composite.

Bioactive glasses and bioactive glass-ceramics are characterized by their ability to form a direct bond with bone. In small particle size ranges (<90 μm), these materials have also been reported to be antibacterial, however, are not optimal for inducing bone formation. Until now, the synergistic nature of a bioactive glass material that has both optimal antibacterial properties and is capable of inducing bone formation (termed “dual action” throughout this document) has not been explored. Furthermore, a porous composite implant that is flexible and pliable and includes a synergistic bioactive glass of this type, heretofore, has not been developed.

In one embodiment, an implant that includes antibacterial, bone-bonding and bone inducing properties is desirable. By incorporating bioactive glass into a porous substrate comprised of silver, beta-tricalcium phosphate and collagen, a porous composite material is formed that is pliable and which has antibacterial properties. By incorporating bioactive glass into a porous substrate comprised of silver, beta-tricalcium phosphate and collagen in a specified bimodal particle size range, a porous composite material is formed that is pliable and which has antibacterial properties and bioactive properties that lead to appropriate bone formation (e.g., bone formation concurrent with implant resorption). It has been particularly determined that a composite material having bioactive glass in a bimodal particle size range distribution that includes both 1) about less than or equal to 53 μm glass particles and 2) from about 90 μm to about 150 μm glass particles facilitates bone growth and prevents surgical site invention.

The bioactive glass used in the present invention may be any alkali-containing ceramic (glass, glass-ceramic, or crystalline) material that reacts as it comes in contact with physiological fluids including, but not limited to, blood and serum, which leads to bone formation. In preferred embodiments, the bioactive glasses, when placed in physiologic fluids, form an apatite layer on their surface. As used herein, “bioactive” relates to the chemical formation of a calcium phosphate layer (amorphous, partially crystalline, or crystalline) via ion exchange between surrounding fluid and the composite material. Bioactive also describes materials that, when subjected to intracorporeal implantation, elicit a reaction. Such a reaction leads to bone formation, attachment into or adjacent to the implant, and/or bone formation or apposition directly to the implant, usually without intervening fibrous tissue.

Preferably, the bioactive glass component of the present invention comprises regions of Combeite crystallite morphology. Such bioactive glass is referred to in this document as “Combeite glass-ceramic”. Examples of preferred bioactive glasses suitable for use in the present invention are described in U.S. Pat. No. 5,681,872 and U.S. Pat. No. 5,914,356, each of which is incorporated by reference in this document in its entirety. Other suitable bioactive materials include 45S5 glass and compositions comprising calcium-phosphorous-sodium silicate and calcium-phosphorous silicate. Further bioactive glass compositions that may be suitable for use in the present invention are described in U.S. Pat. No. 6,709,744, incorporated in this document by reference. Additionally, bioactive materials such as borosilicate, silica, borate, phosphate-containing materials and Wollastonite may also be used. It is understood that some non-alkali-containing bioactive glass materials are within the spirit of the invention. For example, non-alkali containing glass may be substituted for one or both of the bimodal particle size ranges. In certain embodiments, the larger particle size material (90 μm-150 μm) may be, for instance, borosilicate, silica, or Wollastonite bioactive glass. In some embodiments, the material contains another antibacterial agent, as part of its composition. In some embodiments, the materials contain another antibacterial agent, rather than an alkali agent. It should be understood that multiple combinations of alkali and non-alkali containing materials are possible, while maintaining the dual action of antibacterial properties and bone healing properties afforded by a bimodal particle size distribution. Bioactive glasses, as defined in this document, do not include calcium phosphate materials, for example, hydroxyapatite and tricalcium phosphate. However, in addition to bioactive glass, the bioactive antibacterial composition of the present invention may additionally include other materials such as calcium phosphate materials.

In preferred embodiments of the present invention, the bioactive glass is Combeite glass-ceramic (also referred to as “Combeite”). Combeite is a mineral having the chemical composition Na₄Ca₃Si₆O₁₆(OH)₂. It has been found that the use of bioactive glass in restorative compositions, which bioactive glasses include Combeite crystallites in a glass-ceramic structure (hence, Combeite glass-ceramic), in accordance with the present invention gives rise to superior spinal, orthopaedic and dental restorations.

It is preferred that the Combeite glass-ceramic particles which form some or all of the bioactive glass component of the present invention comprise at least about 2% by volume of Combeite crystallites. Combeite glass-ceramic particles containing higher percentages of crystallites are more preferred and volume percentage from about 5% to about 50% of crystallites are particularly desired. It will be appreciated that the Combeite glass-ceramic particles of the present invention are heterogeneous in that they comprise a glassy, amorphous structure having crystallites or regions of Combeite crystallinity dispersed throughout the material.

In one embodiment of the present invention, the heterogeneous particles of Combeite glass-ceramic have average particle sizes of from less than about 150 μm. In another embodiment, the heterogenous particles of Combeite glass-ceramic have average particle sizes of from less than about 150 μm, while still maintaining at least two distinct particle size distributions. In other embodiments of the present invention, two particular Combeite glass-ceramic average particle size ranges have been found to be preferred, in combination, when practiced with the present invention. In one embodiment, the first average particle size range is less than or equal to about 53 μm and the second average particle size range is from about 90 μm to about 150 μm. In another embodiment, the first range is less than or equal to about 90 μm and the second average particle size range is from about 90 μm to abou t 150 μm. In a further embodiment, the first average particle size range is from about 32 μm to about 90 μm and the second average particle size range is from about 90 μm to about 150 μm. The combination of these two ranges practiced together with the present invention is referred to throughout this application as the bimodal particle size range and/or bimodal particle size distribution.

In another embodiment of the present invention, the heterogeneous particles of Combeite glass-ceramic have average particle sizes of from less than about 150 μm. In one embodiment, the heterogenous particles of Combeite glass-ceramic have average particle sizes of from less than about 150 μm, while still maintaining at least two distinct particle size distributions. In other embodiments of the present invention, two particular Combeite glass-ceramic average particle size ranges have been found to be preferred, in combination, when practiced with the present invention. The first range is from about 32 μm to about 90 μm. The second average particle size range is from about 90 μm to about 150 μm. The combination of these two ranges practiced together with the present invention is referred to throughout this application as the bimodal particle size range and/or bimodal particle size distribution.

Methods of determining particle sizes are known in the art. Some methods include passing the particles through several sieves to determine general particle size ranges. Other methods include laser light scattering, and still others are known to persons skilled in the art. Determination of particle size is conveniently accomplished by sieving and such may be used here. Particle size may also be determined via SEM image analysis. It will be appreciated that recitation of averages or size ranges is not meant to exclude every particle with a slightly higher or lower dimension. Rather, sizes of particles are defined practically and in the context of this invention.

In accordance with some preferred embodiments, blends of Combeite glass-ceramics may be useful as the bioactive glass component of the present invention. Thus, a number of different Combeite glass-ceramics can be prepared having different properties, such as Combeite crystallite size, percentage of Combeite crystallites, and the like. It is also preferred in some cases to admix Combeite glass-ceramic in accordance with the present invention with other agents which are consistent with the objectives to be obtained. Thus, a wide variety of such other materials may be so employed so long as composition of the invention comprises bioactive glass equaling at least about 5% by weight of the composition.

In some embodiments, the bioactive glass component may be in the form of fibers, whiskers or strands. In some embodiments, the diameters of these fibers and strands are also bimodal with a first average diameter size of less than or equal to about 53 μm and a second average diameter size range from about 90 μm to about 150 μm. In other embodiments, the diameters of these fibers and strands are also bimodal with a first average diameter size of about 32 μm to about 90 μm and a second average diameter size range from about 90 μm to about 150 μm.

Certain antibacterial compositions described herein comprise silver. In some embodiments, silver is furnished to the composition by the addition or incorporation of a silver salt. Non-limiting examples of silver salts useful in the compositions of the invention include Ag₂O and Ag₂NO₃. In various embodiments, silver comprises about 1-20% by weight of the antibacterial composition of the invention. In specific embodiments, silver comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of the antibacterial composition of the invention.

In some embodiments, the bioactive glass comprises at least one alkali metal such as, for example, lithium, sodium, potassium, rubidium, cesium, francium, or combinations of these metals. In other embodiments, however, the bioactive glass has little to no alkali metal. For example, in certain embodiments, the bioactive glass has 30% or less of alkali metal. In other embodiments, the bioactive glass has 25% or less of alkali metal. In yet other embodiments, the bioactive glass has 20% or less of alkali metal. In yet other embodiments, the bioactive glass has 15% or less of alkali metal. In other embodiments, the bioactive glass has 10% or less of alkali metal. In still other embodiments, the bioactive glass has 5% or less of alkali metal. In yet other embodiments, the bioactive glass has substantially no alkali metal. However, in these embodiments an antibacterial agent, for example silver, may be substituted for the alkali metal.

In exemplary embodiments of the present invention, the bioactive glass also has osteoproductive properties. As used in this document, “osteoproductive” refers to an ability to allow osteoblasts to proliferate, allowing bone to regenerate. “Osteoproductive” may also be defined as conducive to a process in which a bioactive surface is colonized by osteogenic stem cells and which results in more rapid filling of defects than that produced by merely osteoconductive materials. Combeite glass-ceramic is an example of an osteoproductive, bioactive material.

According to one embodiment of the present invention, the composite material may comprise up to about 80% of the bioactive glass. In some embodiments having a bimodal particle size distribution, about 50% by weight of this glass having a particle size range of less than or equal to about 53 μm and about 50% by weight having a second average diameter size range from about 90 μm to about 150 μm. In some embodiments having a bimodal particle size distribution, about 50% by weight of this glass having a particle size range of about 32 μm to about 90 μm and about 50% by weight having a second average diameter size range from about 90 iim to about 150 μm. In certain embodiments, the bimodal bioactive glass is present in an amount of about 10 to 50% by weight of the composite material.

In other embodiments, the present invention material is comprised entirely of bioactive glass (e.g. 100% by weight).

In particular embodiments having a bimodal bioactive glass particle size distribution, the relative percentage of small and large bioactive glass particles within the present invention may be tailored based on the desired antibacterial efficacy and bone formation. In preferred embodiments of the present invention, about 50% by weight of the bioactive glass particles are from the smaller distribution and about 50% by weight of the bioactive glass particles are from the larger distribution. In some embodiments, the percentage of small bioactive glass particles may be less than about 25% by weight of the total amount of bioactive glass and the large bioactive glass particles may be about 75% or more by weight of the total amount of bioactive glass. In yet other embodiments, the percentage of small particles may be about 75% or more by weight of the total amount of bioactive glass while the percentage of large particles may be about 25% or less by weight of the total amount of bioactive glass. For instance, the present invention may be tailored to provide greater antibacterial efficacy in compromised sites by increasing the percentage of small particle size glass and correspondingly decreasing the amount of large particle size glass.

The bimodal bioactive glass particle size distribution of some embodiments described herein conveys benefits. While not being bound by a specific mechanism of action, it is believed that the high surface area of the small particles (e.g., <53 μm or 32 μm-90 μm), results in an early burst release of ions and a quick resorption (e.g., 4 weeks or less) that is ideal for the materials and composites of the present invention. Specifically, the antibacterial effect of the small particles commences immediately and continues several weeks until the bacteria are effectively killed off. At this time, the small particles completely resorb, are absent from the site of healing and make way for cells and vessels that aid in bone healing. The large particles (90 μm-150 μm) with lower surface area, more slowly release the ions that create an environment which stimulates osteoblasts for the formation of bone. The bone grows over time as the larger glass particles resorb, however, the large particles are able to provide a substrate for the bone growth prior to resorbing (e.g., 24-52 weeks).

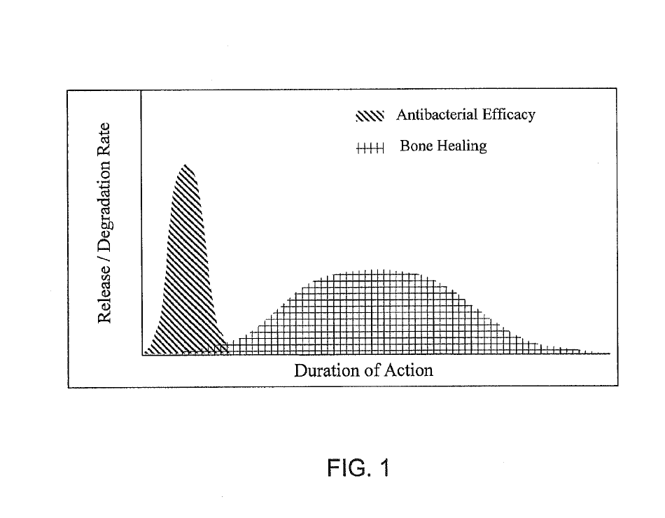

FIG. 1 is a graphical depiction of the in-vivo ion release and degradation profile of the bioactive antibacterial material of the present invention. The profile demonstrates the bimodal particle size distribution with the smaller particle sizes (<53 μm) displaying faster resorption kinetics than the larger particle size (90 μm-150 μm). As described above, the increased surface area of the small particles leads to burst release of ions. In contrast, the larger particles have a slower dissolution rate and therefore require a longer period of time for complete resorption. This larger particle size range also lends itself to partial dissolution and partial cell-mediated resorption.

In certain embodiments, the present invention comprises calcium phosphate having macroporosity, mesoporosity, and microporosity. More preferably, the porosity of the calcium phosphate is interconnected and highly porous. The preparation of preferred forms of calcium phosphate for use in the present invention is described in U.S. Pat. No. 6,383,519 and U.S. Pat. No. 6,521,246, incorporated into this application by reference in their entireties. An exemplary calcium phosphate product is Vitoss® Bone Graft Substitute (available from Orthovita, Inc. of Malvern, Pa.).

The present invention composite may be formed into a variety of shapes or may be cut or shaped at the time of surgery. In other embodiments, the bioactive composite implant is used to fill cavities of metal or non-resorbable implants. For instance, when used with a shaped spinal implant, the present invention composite material may be present within the center cavity of the implant to convey antibacterial activity and to facilitate fusion of the adjacent vertebral bodies.

In a preferred embodiment of the present invention, a bioactive antibacterial composite is formed upon combining silver with a resorbable biocompatible polymer, resorbable calcium phosphate and resorbable bioactive glass as described in the present invention.

The biocompatible polymer used in the present invention is preferably a natural polymer. Examples of natural biocompatible polymers that are suitable for use in the present invention alone or in combination include collagen and similar organic biomaterials and natural biocompatible polymers. Suitable collagens are described, for example, in U.S. Pat. No. 7,189,263, which is herein incorporated by reference in its entirety. Some embodiments of the present invention contain collagen that comprises up to 100% Type I collagen. In other embodiments, the collagens used may be predominantly, or up to about 90%, of Type I collagen with up to about 5% of Type III collagen or up to about 5% of other types of collagen. Suitable Type I collagens include native fibrous insoluble human, bovine, porcine, or synthetic collagen, soluble collagen, reconstituted collagen, or combinations thereof.

In a preferred embodiment of the present invention, the biocompatible polymer is Type I bovine collagen; and the calcium phosphate is tricalcium phosphate and, more preferably beta-tricalcium phosphate, with a total porosity of at least about 30% and a particle size range of from about 0.25 mm to about 2 mm. Porous calcium phosphate morsels to be used with the present invention are preferably greater than about 0.25 mm in size. The morsels of calcium phosphate may be about 1-2 mm in size for some embodiments of the present invention. The calcium phosphate morsels may be about 0.25 mm to about 1 mm or to about 2 mm for other embodiments of the present invention. For flowable compositions of the present invention, it will be appreciated that the morsel size will be selected considering the desired delivery apparatus. For example, for delivery of a flowable composition using a standard syringe, it will be necessary to select a morsel size that fits through the syringe orifice. Selection of the appropriate morsel size is believed to be with the capability of the skilled artisan.

In some embodiments, the bioactive antibacterial composite of the present invention will comprise about 1-20% by weight of silver; about 10-80% by weight of calcium phosphate; about 5-20% by weight of collagen; and about 5-80% by weight of bioactive glass. In other embodiments, the bone graft materials of the present invention will comprise about 1-15% by weight of silver; about 50-90% by weight of calcium phosphate; about 5-25% by weight of collagen, and about 5-40% by weight of bioactive glass. In certain embodiments, bone graft materials of the present invention comprise silver in a composition comprising calcium phosphate, collagen, and bimodal bioactive glass having a weight ratio of about 70:20:10. In other embodiments, the weight ratio of calcium phosphate, collagen, and bioactive glass is about 80:10:10. In yet others, the weight ratio of calcium phosphate, collagen, and bioactive glass is about 80:15:5. In further embodiments, the weight ratio of calcium phosphate, collagen, and bioactive glass is about 50-55 calcium phosphate:10-15 collagen:30-40 bioactive glass. In others, the weight ratio of calcium phosphate, collagen, and bioactive glass is about 10:10:80. The weight ratio of the calcium phosphate, collagen, and bioactive glass may also be about 60:20:20. In a preferred embodiment, the weight ratio of the calcium phosphate, collagen, and bioactive glass is about 65:15:20. The mass ratios may be altered without unreasonable testing using methods readily available in the art while still maintaining all the properties that attribute to an effective bone graft. One unique feature of the bone graft materials of the present invention is that the mineral remains porous even when combined with the collagen and bioactive glass. Further, the resultant composite bone graft is itself highly porous with a broad pore size distribution as described herein.

The use of collagen has been determined to provide flexible, pliable, or flowable handling properties to the composite so that in addition to being antibacterial, osteoproductive and bioactive, the composite bone graft can also be manipulated, for example, wrapped, cut, bended, and/or shaped, particularly when wetted to fill defects of various sizes. In addition, the porosity of the calcium phosphate imparts porosity to the composite that enables bone growth to occur concurrent with implant resorption by promoting capillary action of fluids, allowing recruitment of cells for bone formation, and permitting angiogenesis.

In preferred embodiments, the composite material comprises varying levels of pore sizes that are interconnected. In exemplary embodiments of the invention, the bone grafts comprise three different porosity size ranges, herein described as macroporosity, mesoporosity, and microporosity. Preferably, the macroporosity, mesoporosity, and microporosity occurs simultaneously. Within the scope of this invention, macroporosity is defined as having pore diameters greater than or equal to 100 microns. Mesoporosity is defined as having a pore diameter less than 100 microns but greater than or equal to 10 microns. Microporosity is defined as having a pore diameter less than 10 microns.

Persons skilled in the art can easily determine whether a material has each type of porosity through examination, such as through the preferred method of scanning electron microscopy. While it is certainly true that more than one or a few pores within the requisite size range are needed in order to characterize a sample as having a substantial degree of that particular form of porosity, no specific number of percentage is called for. Rather, a qualitative evaluation by persons skilled in the art shall be used to determine macroporosity, mesoporosity, and microporosity.

While the invention does not require a specific percentage for each of the three porosity size ranges described, certain percentages of each porosity size range have been found to be particularly well suited for bone graft materials of the present invention. For example, in certain embodiments, the bone graft materials can be characterized as having about 10-25% of the pores within the microporosity range; about 50-70% of the pores within the mesoporosity range; and about 10-30% of the pores within the macroporosity range.

It will be appreciated that in some embodiments, the overall porosity of materials prepared in accordance with this invention is high. This characteristic is measured by pore volume, expressed as a percentage. Zero percent pore volume refers to a fully dense material, which, perforce, has no pores at all. One hundred percent pore volume cannot meaningfully exist since the same would refer to “all pores” or air. Persons skilled in the art understand the concept of pore volume, however and can easily calculate and apply it. For example, pore volume may be determined in accordance with Kingery, W. D., Introduction to Ceramics, Wiley Series on the Science and Technology of Materials, 1^{st} Ed., Hollowman, J. H., et al. (Eds.), Wiley & Sons, 1960, p. 409-417, who provides a formula for determination of porosity. Expressing porosity as a percentage yields pore volume. The formula is: Pore Volume=(1−fₚ) 100%, where fₚ is fraction of theoretical density achieved.

Porosity can be measured by methods known in the art such as helium pycnometry. This procedure determines the density and true volume of a sample by measuring the pressure change of helium in a calibrated volume. A sample of known weight and dimensions is placed in the pycnometer, which determines density and volume. From the sample's mass, the pycnometer determines true density and volume. From measured dimensions, apparent density and volume can be determined. Porosity of the sample is then calculated using (apparent volume−measured volume)/apparent volume. Porosity and pore size distribution may also be measured by mercury intrusion porosimetry, another method known in the art.

Pore volumes in excess of about 30% may be achieved in accordance with this invention while materials having pore volumes in excess of 50% or 60% may also be routinely attainable. Some embodiments of the invention may have pore volumes of at least about 70%. Other embodiments have pore volumes in excess of about 75% or about 80%. Pore volumes greater than about 85% are possible, as are volumes of about 90%. In preferred cases, such high pore volumes are attained while also attaining the presence of interconnected macro-meso-, and microporosity as well as physical stability of the materials produced. It is believed to be a great advantage to prepare graft materials having macro-, meso-, and microporosity simultaneously with high pore volumes that also retain some compression resistance and flexibility, moldability, or flowability when wetted.

Due to the high porosity and broad pore size distribution of the present invention composite graft, the implant is not only able to wick/soak/imbibe materials very quickly, but is also capable of retaining them. A variety of fluids could be used with the present invention including blood, bone marrow aspirate, saline, antibiotics and proteins such as bone morphogenetic proteins (BMPs). Materials of the present invention can also be imbibed with cells (e.g., fibroblasts, mesenchymal, stromal, marrow and stem cells), platelet rich plasma, other biological fluids, and any combination of the above. Bone grafts of the present invention actually hold, maintain, and/or retain fluids once they are imbibed, allowing for contained, localized delivery of imbibed fluids and rapid release of ions from the bioactive glass. In this manner, fluids activate the present invention bioactive glass material. This capability has utility in cell-seeding, drug delivery, and delivery of biologic molecules as well as in the application of bone tissue engineering, orthopaedics, and carriers of pharmaceuticals.

Bioactive antibacterial composites and shaped bodies of the present invention made from the composites preferably demonstrate properties suitable for use in spinal and orthopaedic procedures. Bioactive antibacterial composites and shaped bodies of the present invention made from the composites also preferably demonstrate bioactivity. A formed bioactive composite material according to the present invention can be placed in or near bone voids to facilitate new bone growth while preventing surgical site infection. After some time in the body, the implanted material will begin to bridge the void or facilitate fusion of adjacent bony structures thereby restoring and repairing the site.

It will be appreciated by those skilled in the art that the silver-containing bioactive antibacterial composites of the present invention may be used in a wide variety of restorative and surgical procedures. One example is the repair or fusion of vertebrae of the spine. Lower back pain may oftentimes be attributed to the rupture or degeneration of lumbar intervertebral discs due to degenerative disc disease, ischemic spondylolisthesis, post laminectomy syndrome, deformative disorders, trauma, tumors and the like. This pain may result from the compression of spinal nerve roots by damaged discs between the vertebra, the collapse of the disc, and the resulting adverse effects of bearing the majority of the patient's body weight through a damaged unstable vertebral joint. To remedy this, spinal implants may be inserted between the vertebral bodies to stabilize and support the joint and facilitate fusion via bone bonding. To facilitate the fusion, bone graft substitute materials such as the material of the present invention are placed in or around the spinal implant to facilitate bone ingrowth prior to resorbing. It is envisioned that the present invention composite material would serve well as a bone graft substitute.

In other embodiments of the present invention, the composite shaped body may be used in a variety of orthopaedic procedures involving bone repair and restoration. The present invention composite may be formed into a sleeve or cup. The silver-containing bioactive antibacterial composite of the present invention may also be used in conjunction with orthopaedic appliances such as joints, rods, pins, suture fasteners, anchors, repair devices, rivets, staples, tacks, orthopaedic screws and interference screws. Such bioactive antibacterial composite shaped bodies can be used in conjunction with biocompatible gels, pastes, cements or fluids and surgical techniques that are known in the art. Thus, a shaped body comprised of the present invention composite material can be inserted into bone and the bioactivity and antibacterial properties of the material will give rise to osteogenesis and beneficial medical or surgical results.

Many of the embodiments disclosed herein are to fill bony voids and defects. It will be appreciated that applications for the embodiments of the present invention include, but are not limited to, filling interbody fusion devices/cages (ring cages, cylindrical cages), placement adjacent to cages (i.e., in front of cages), placement in the posterolateral gutters in posterolateral fusion (PLF) procedures, backfilling the iliac crest, acetabular reconstruction and revision hips and knees, large tumor voids, use in high tibial osteotomy, burr hole filling, and use in other cranial defects. The bone graft material strips may be suited for use in PLF by placement in the posterolateral gutters, and in onlay fusion grafting. Additional uses may include craniofacial and trauma procedures that require covering or wrapping of the injured/void site. The bone graft material cylinders may be suited to fill spinal cages and large bone voids, and for placement along the posterolateral gutters in the spine.

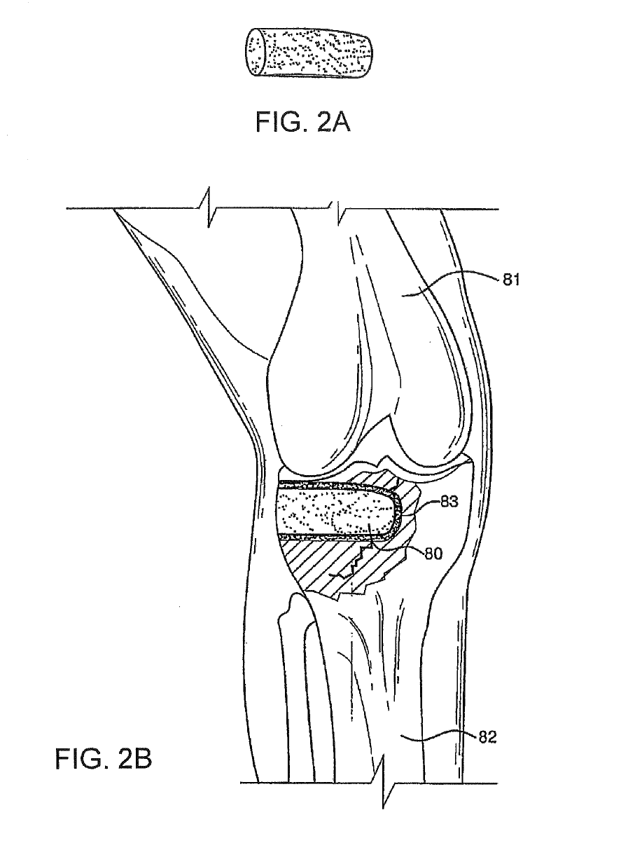

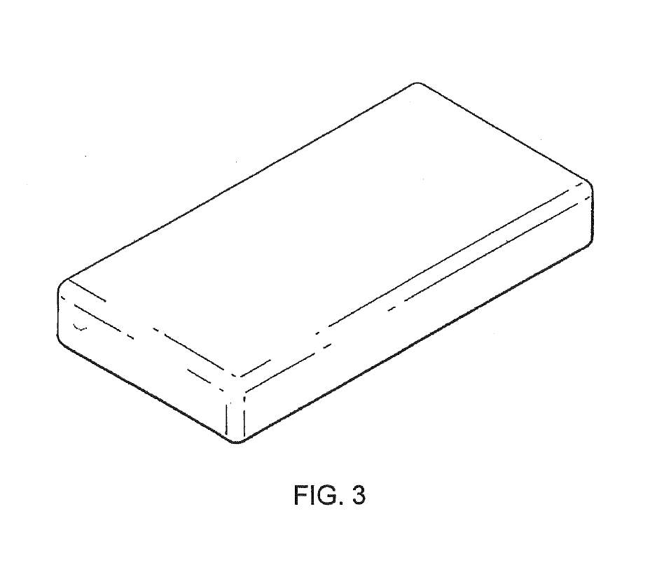

Due to the wide range of applications for the embodiments of the present invention, it should be understood that the present invention graft material could be made in a wide variety of shapes and sizes via standard molding techniques. For instance, blocks and cylinders of the present invention may find utility in bone void filling and filling of interbody fusion devices; wedge shaped devices of the present invention may find utility in high tibial osteotomies; and strips may find utility in cranial defect repairs. FIGS. 2A and 2B show the material of the present invention within a human tibia that is used as a block for bulk restoration or repair of bulk defects in bone or oncology defects.

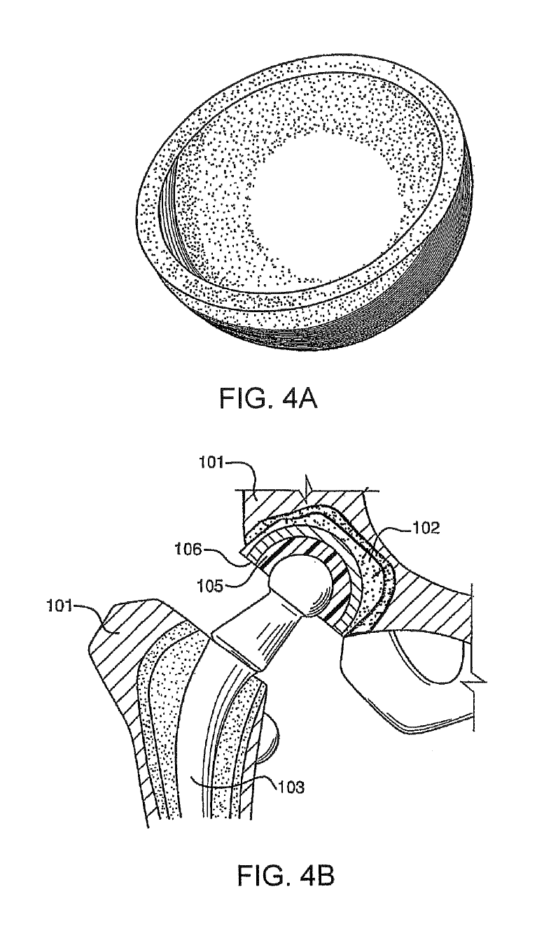

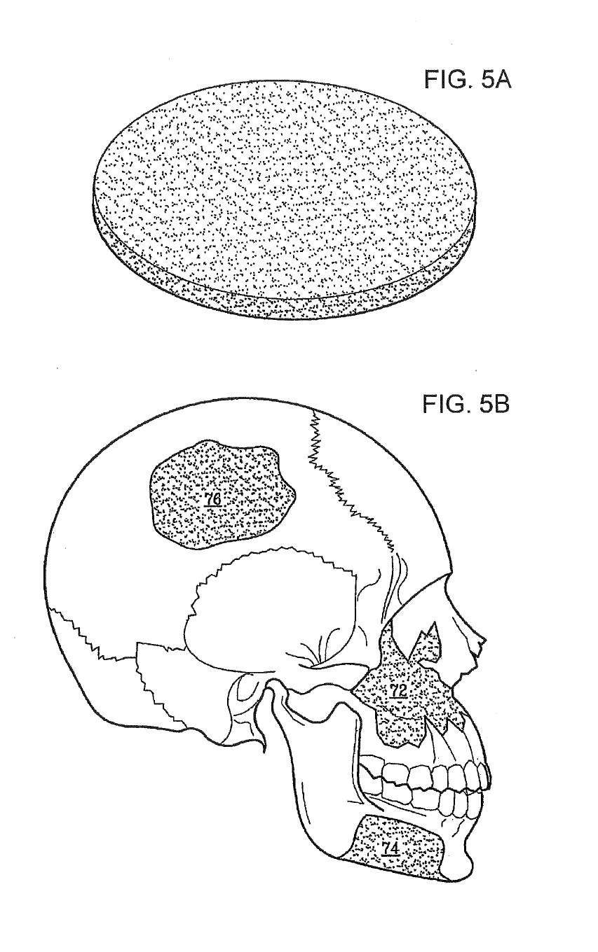

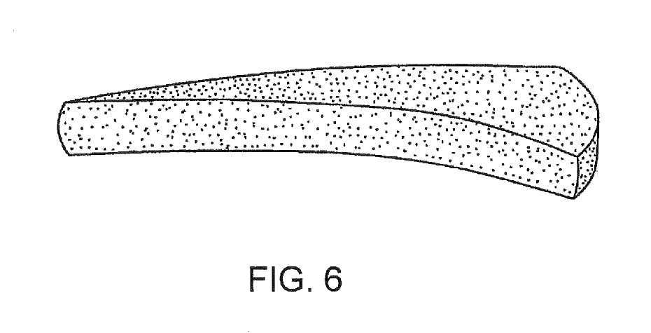

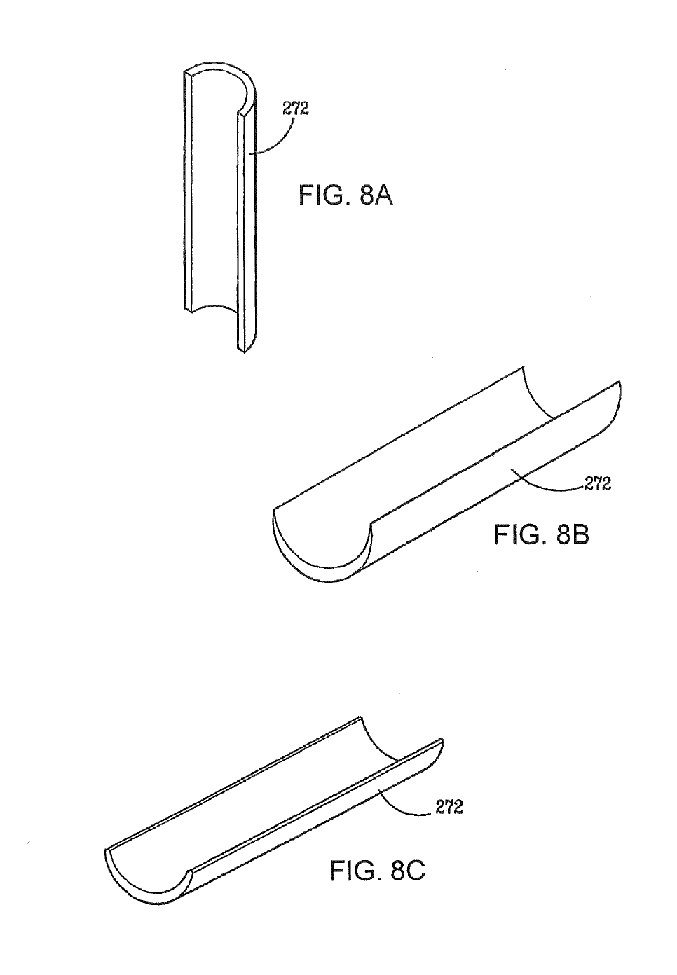

Of particular interest, may be the use of some of the graft materials as semi-spherical (FIG. 4A), semi-tubular (FIGS. 8A-8C) or disc-shaped (FIG. 5A) strips for graft containment devices. An embodiment of the semi-spherical form 102 in use is depicted in FIG. 4B.

It will be appreciated that these shapes are not intended to limit the scope of the invention as modifications to these shapes may occur to fulfill the needs of one skilled in the art. The benefits of the graft containment materials that, for instance, may be used in acetabular reconstruction made from the present invention are several-fold. The graft materials may act as both a barrier to prevent migration of other implants or graft materials and serves as an osteoconductive resorbable bone graft capable of promoting bone formation. The graft containment device may be relatively non-load-bearing, or partially load-bearing, or may be reinforced to be fully load-bearing as described below. Depending on the form, the graft materials have barrier properties because it maintains its structural integrity.

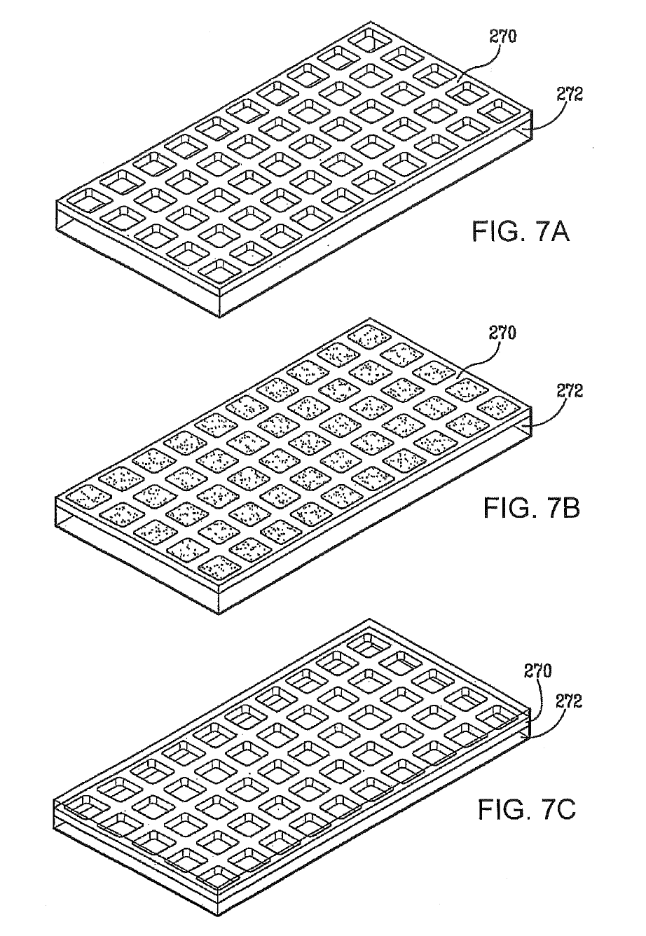

In applications requiring graft materials with load-bearing capabilities, the graft materials of the present invention may have meshes or plates affixed. The meshes or plates may be of metal, such as titanium or stainless steel, or of a polymer or composite polymer such as polyetheretherketone (PEEK), or nitinol. As depicted in FIGS. 7A and 7B, a metallic mesh 270 may be placed to one side of the bone graft material 272 to add strength and load-bearing properties to the implant. In FIG. 7A, the mesh plate 270 sits affixed to one surface of the graft material 272. In FIG. 7B, the mesh plate 270 penetrates one surface of the graft material 272 with one side of mesh exposed on top. In FIG. 7C, the mesh plate 270 is immersed more deeply than in FIG. 7B within the graft material 272. FIGS. 8A-8C depict another embodiment of the graft material 272 in semi-tubular form. A mesh may be affixed to a surface for further support in long bone reinforcement. Due to the unique properties of the present invention graft material, the mesh may be affixed in the body using sutures, staples, screws, cerclage wire or the like.

One skilled in the art may place the mesh in any location necessary for a selected procedure in a selected bodily void. For instance, a composite of mesh and graft material could be used in a craniomaxillofacial skull defect with the more pliable graft surface being placed in closer proximity to the brain and the more resilient mesh surface mating with the resilient cortical bone of the skull. In this manner, the mesh or plate may be affixed to one side of the graft material. Alternatively, the mesh or plate may be affixed to both sides of the graft material in sandwich fashion. Likewise, graft material could be affixed to both sides of the mesh or plate. In some embodiments, the mesh may be immersed within the graft material. The meshes may be flat or may be shaped to outline the graft material such as in a semi-spherical, semi-tubular, or custom form. These embodiments may be unique due to their integral relation between the graft material and the mesh. This is contrary to other products in the field in which the graft material is placed adjacent to the structural implant or, in the case of a cage, within the implant.

In accordance with the present invention, another embodiment provides a bone graft for long bone reinforcement comprising a biocompatible, resorbable semi-tubular shape, or sleeve, of β-tricalcium phosphate, collagen, silver, and bioactive glass, the entire graft having interconnected macro-, meso-, and microporosity. A mesh may be affixed to the surface of the sleeve or may be immersed in the sleeve. The mesh may be made of titanium, stainless steel, nitinol, a composite polymer, or polyetheretherketone. The cross-section of the sleeve may be in the shape of a crescent shape moon (FIG. 8B).

In other embodiments, there is a graft for the restoration of bone in the form of a shaped body, the shaped body comprising β-tricalcium phosphate, collagen, silver, and bioactive glass, the material of the graft having interconnected macro-, meso-, and microporosity; the body shape being selected to conform generally to a mammalian, anatomical bone structure. The shapes will vary depending on the area of the body being repaired. Some basic shapes may be a disk, semi-sphere, semi-tubular, or torus. In some embodiments, the shape will conform generally to the acetabulum.

Other graft materials of the present invention having load-bearing capabilities may be open framed, such that the bone graft material is embedded in the central opening of the frame. The frame may be made of a metal such as titanium or of a load-bearing resorbable composite such as PEEK or a composite of some form of poly-lactic acid (PLA). In the case of the latter, the acid from the PLA co-acts, or interacts with the calcium phosphate of the embedded bone graft material to provide an implant with superior resorption features.

The graft materials can also be imbibed with any bioabsorbable polymer or film-forming agent such as polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), polyesters and the like. The resultant graft material is strong, carveable, and compressible. The grafts of the present invention coated with agents such as the aforementioned may still absorb blood.

In another embodiment of the present invention, the graft materials may be used as an attachment or coating to any orthopaedic implant such as a metal hip stem, acetabular component, humeral or metatarsal implant, vertebral body replacement device, pedicle screw, general fixation screw, plate or the like. The coating may be formed by dipping or suspending the implant for a period of time in a substantially homogenous slurry of calcium phosphate, collagen, silver, and bioactive glass and then processing via freeze-drying/lypholization and crosslinking techniques. As used in this context, substantially homogenous means that the ratio of elements within the slurry is the same throughout. Alternatively, a female mold may be made of the implant and the slurry may be poured into the mold and processed, as described above, to form the coating.

In yet another embodiment of the present invention, the graft material may be shredded or cut into small pieces. These smaller shredded pieces could then be used as filler or could be placed in a syringe body. In this fashion, fluids could be directly aspirated into or injected into the syringe body thereby forming a cohesive, shapeable bone graft mass “in situ” depending upon the application requirements. The shredded pieces find particular use as filler for irregular bone void defects. Further, unlike traditional bone graft substitutes they are highly compressible and therefore can be packed/impacted to insure maximum contact with adjacent bone for beneficial healing.

The bioactive composite of the present invention may also find particular utility in a variety of dental bone grafting procedures.

The collagen, silver and bioactive glass may be combined with the calcium phosphate by blending to form a substantially homogenous mixture. As used in this context, substantially homogenous means that the ratio of components within the mixture is the same throughout. The calcium phosphate, collagen, silver, and bioactive glass may also be combined to form a composite matrix in various shapes and sizes. In certain embodiments, the bioactive glass could be in the form of a coating on the collagen strands. In others, the bioactive glass could be in the form of a coating on a collagen and calcium phosphate homogenous mixture. Upon treatment using various preferred heating, freeze-drying, and crosslinking techniques, such mixtures of the present invention form graft materials that may be preferred. In one method, the three constituents (the inorganic calcium phosphate component, collagen, and bioactive glass), are mixed while the pH of the homogenate is monitored. The bioactive component is sensitive to aqueous environments, so monitoring the pH of the homogenate ensures that the bioactive glass component in the mix is not altered via premature leaching of ions that are necessary for promoting osteoactivity and bioactivity and for maintaining antibacterial properties. The homogenate is then dispersed into defined molds, freeze-dried, and for some embodiments, crosslinked.

In another method, the collagen and the inorganic component are combined as described, and the silver-containing bioactive glass is provided as a distinct component, to be incorporated into the bone graft material during preparation for use in the surgical site. Contemplated herein is a kit comprising a bone graft and silver-containing bioactive glass. The bone graft provided in a kit may comprise collagen and calcium phosphate. In a kit, the bioactive glass may be provided in a unit dose, or two unit doses in which the two distinct glass particle sizes are separated, to be combined with the bone graft provided at the time of surgery. The bioactive glass may be provided in a single container or multiple containers. The components may be mixed together with fluid at the time of surgery to form a pliable, putty-like bioactive antibacterial bone graft substitute.

Certain aspects of the present invention provide for kits that contain sterile shaped implants within sterile packaging alongside appropriate instrumentation for inserting or implanting the shaped implant. For instance, the bone graft provided in a kit may be enclosed in a delivery apparatus, such as a syringe, or, the bone graft may be provided in addition to a syringe capable of holding and delivering the bone graft. Flowable bone graft materials (such as those described in U.S. Patent Application No. 2005/0288795, filed on Jun. 23, 2004, incorporated herein by reference in its entirety) are contemplated as being particularly suitable for such a kit. The bioactive glass may be within the delivery or holding apparatus along with the graft, or the bioactive glass may be provided in a second apparatus, such as a syringe. The bioactive-glass-containing apparatus may be adapted to connect to the bone graft apparatus such that homogenous mixing back and forth is permitted. Thus, ultimately, a composite apparatus capable of mixing the components into a substantially homogenous flexible, pliable bone graft containing calcium phosphate, collagen, silver and bioactive glass is provided.

The shaped bodies can be modified in a number of ways to increase or decrease their physical strength and other properties so as to lend those bodies to still further modes of employment. Overall, the present invention is extraordinarily broad in that shaped bodies may be formed easily and with enormous flexibility. Preformed shapes may be formed in accordance with the invention from which shapes may be cut or formed.

Throughout this disclosure, various aspects of the invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 10 to 40 should be considered to have specifically disclosed subranges such as from 10 to 30, from 10 to 20, from 20 to 40, from 15 to 35, from 13 to 26 etc., as well as individual numbers within that range, for example, 10, 20, 25.5, 30, 31.3, 35, and 40. This applies regardless of the breadth of the range.

### EXAMPLES

Additional objects, advantages, and novel features of this invention will become apparent to those skilled in the art upon examination of the following examples of the invention. The examples are included to more clearly demonstrate the overall nature of the invention. The examples are exemplary, not restrictive, of the invention.

### Example 1

### Scanning Electron Microscopy (SEM) Images of Several Embodiments of the Present Invention

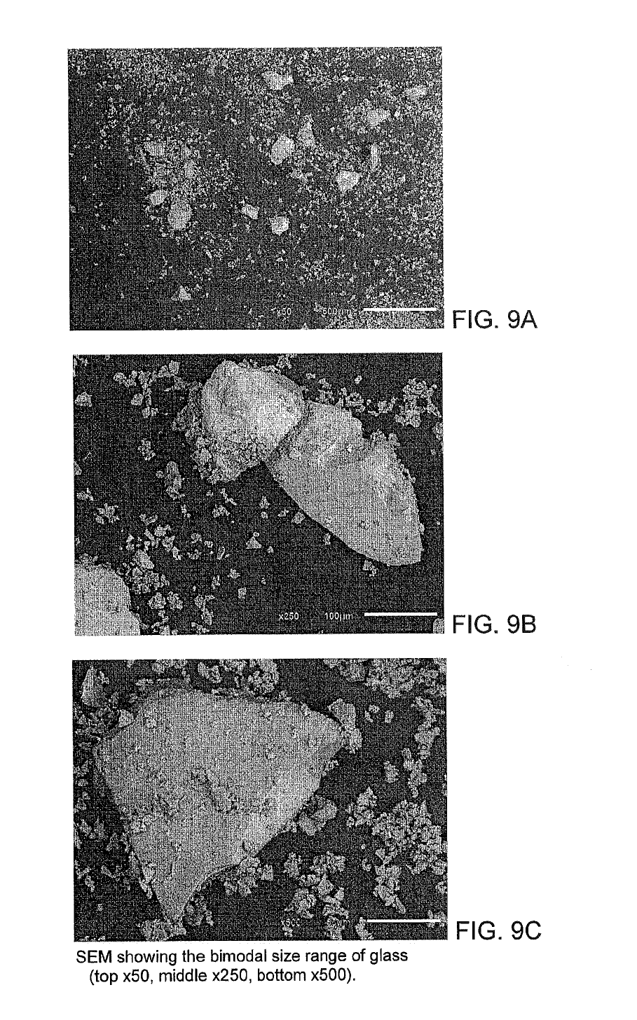

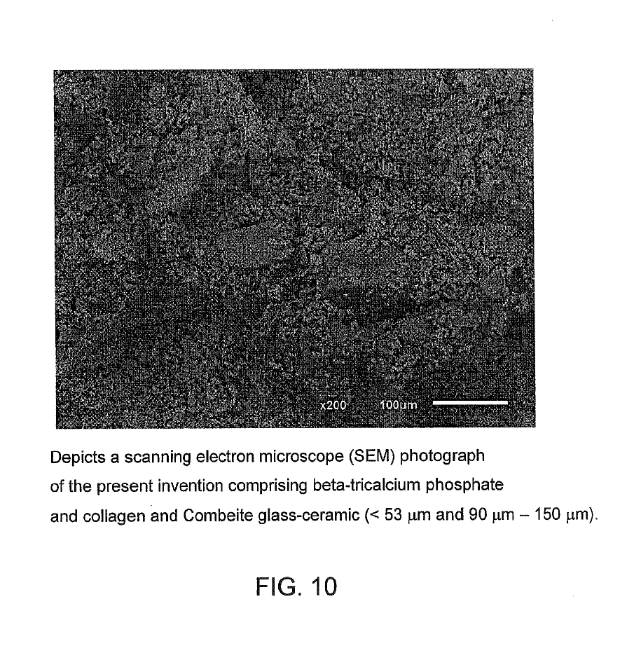

Scanning electron microscopy (SEM) was performed to qualitatively evaluate the bimodal nature of the bioactive glass of the present invention in morsel form and as part of a composite. FIGS. 9A, 9B and 9C are representative SEM images of the present invention showing bimodal Combeite glass-ceramic particles <53 μm in size and from about 90 μm-150 μm in size at three different magnifications—×50 magnification (FIG. 9A), ×250 magnification (FIG. 9B) and at ×500 magnification (FIG. 9C). FIG. 10 is a representative SEM image of another embodiment of the present invention in composite form in which the bimodal Combeite glass-ceramic comprises about 20% by weight of the composite, the beta-tricalcium phosphate comprises about 65% by weight of the composite and collagen comprises about 15% by weight of the composite (×200 magnification).

### Example 2

### Laser Scattering Particle Size Distribution Analyzer Evaluation

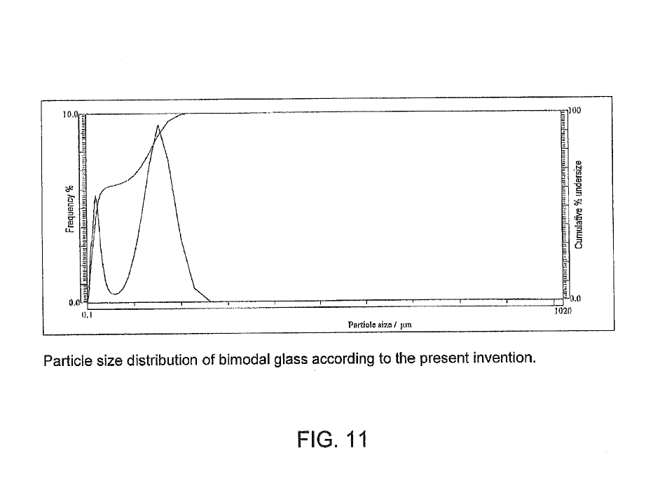

Particle size distribution of the bimodal glass of the present invention was performed using a laser scattering particle size distribution analyzer (Horiba LA-910). This analytical technique provides information on the D10 (particle size for which 10% of the particle size distribution is below this value), D50 (particle size for which 50% of the particle size distribution is below this value) and D90 (particle size for which 90% of the particle size distribution is below this value). Bimodal Combeite glass-ceramic particles (<53 μm in size and from about 90 μm-150 μm in size) were analyzed in triplicate. Approximately 50% of the particles, by weight, were below 53 microns in size while approximately 50% of the particles, by weight, were between 90-150 microns in size. An exemplary particle size distribution graph of the bimodal glass is shown in FIG. 11. The D10 of this particle size distribution was between about 2 and about 6 microns, the D50 was between about 20 and about 25 microns, and the D90 was between about 150 and about 160 microns.

### Example 3

### In-Vitro Bioactivity

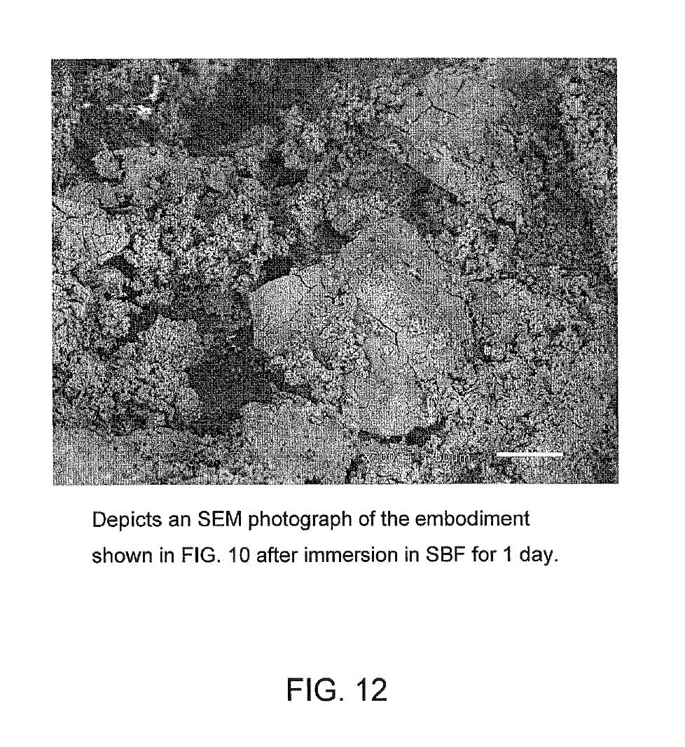

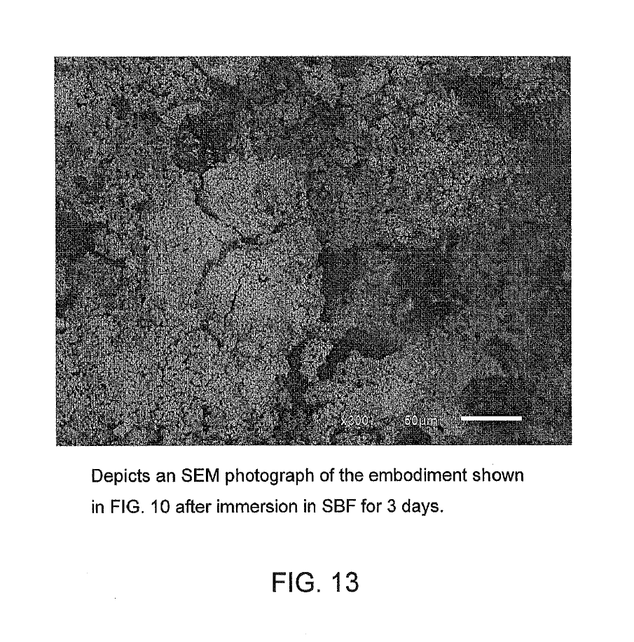

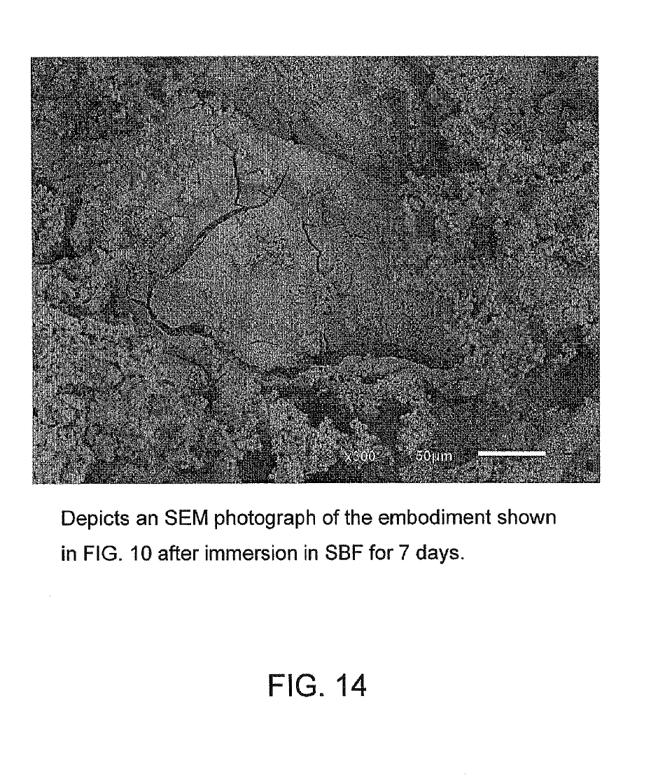

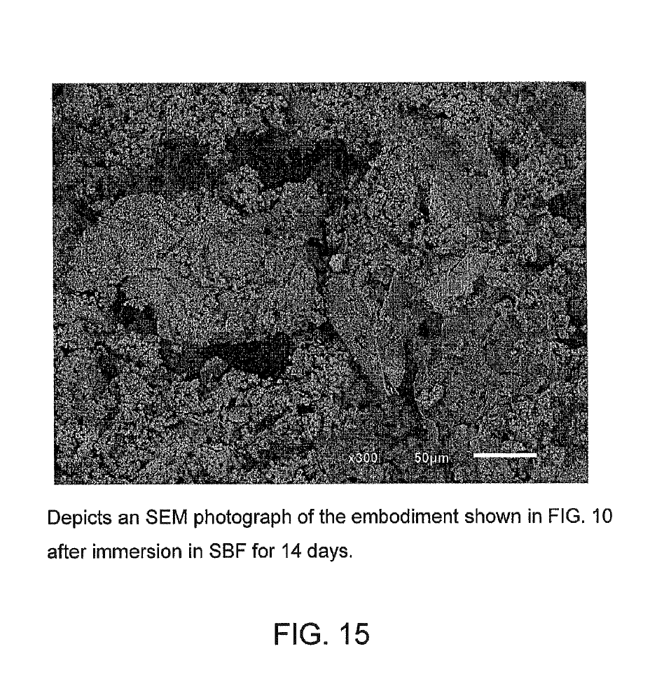

In vitro bioactivity studies were performed with the test materials of the present invention using the method of Kokubo, How useful is SBF in predicting in vivo bone bioactivity, Biomaterials (2006) 27:2907-2915. Samples were made by combining a strip of calcium phosphate and collagen (Vitoss® (VT) Foam Pack Bone Graft Substitute (Orthovita, Inc., Malvern, Pa.)) with bimodal Combeite bioactive glass-ceramic particles. Fifty percent (50%) by weight of the glass particles were about <53 μm in size and 50% by weight of the glass particles were between about 90 -150 μm in size. An amount of saline approximately equal to the volume of strip material was added to the strip along with the glass-ceramic particles, and all materials were kneaded together for approximately 2 minutes to form a putty-like (“pack”) composite material. The final ratio of calcium phosphate: collagen: bimodal glass was approximately 65:15:20. The samples were suspended in simulated body fluid at 37° C. for 1, 3, 7 and 14 days (FIGS. 12, 13, 14 and 15, respectively). After immersion in SBF, the formation of a significant amount of calcium phosphate was observed on the composite material even as early as 1 day.

### Example 4

### Wettability and Compression Resistance

Pliable “pack” samples were made by combining a strip of calcium phosphate and collagen with bimodal Combeite bioactive glass-ceramic particles as described above in Example 3. Fifty percent (50%) by weight of the glass particles were about <53 μm and 50% by weight of the glass particles were between about 90-150 μm in size. Two concentrations of the bimodal Combeite were tested—100 mg of Combeite per mL of bone graft (equal to a calcium phosphate: collagen: bimodal glass ratio of approximately 65:15:20) and 200 mg of Combeite per mL of bone graft (equal to a calcium phosphate: collagen: bimodal glass ratio of approximately 54:14:32). The pack materials were inserted into a syringe and a mechanical testing device was used to apply a constant pressure on the plunger of the syringe, thereby compressing the material being tested, until a pressure of approximately 30 lbf was reached. The weights of the materials before and after compression were recorded and used to calculate the fluid retention capability of the material (Table 1).

TABLE 1

| Bimodal | | | | Hydrated | |
|---|---|---|---|---|---|
| glass | Dry | Hydrated | | weight | |
| concentration | weight | weight | Mass | after com- | Fluid |
| (mg/mL) | (g) | (g) | increase | pression (g) | retention |
| 100 | 2.8154 | 5.8524 | 207.9% | 5.8056 | 99.2% |
| 200 | 3.3274 | 7.2804 | 218.8% | 7.2128 | 99.1% |

### Example 5

### Radiopacity

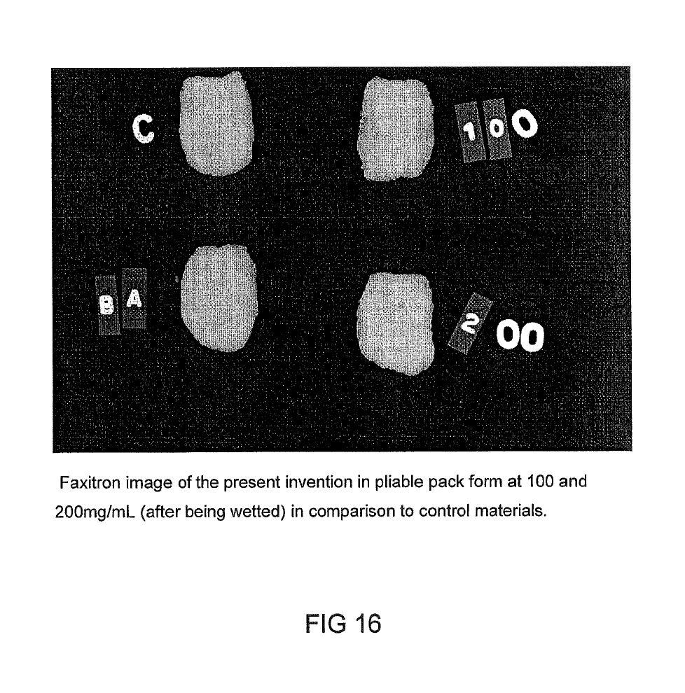

A faxitron high-resolution x-ray was taken of the present invention materials, prepared as described above in Examples 3 and 4. The radiopacity of the present invention embodiments was similar to that of controls that did not contain bioactive glass in a bimodal particle size range (FIG. 16). Note: “C”—VT Foam Pack Bone Graft Substitute (Orthovita, Inc., Malvern, Pa.) control; “BA”—Vitoss™ BA Bioactive Bone Graft Substitute (Orthovita, Inc., Malvern, Pa.) control; “100”—present invention material with 100 mg of bimodal Combeite per mL of bone graft in which fifty percent (50%) by weight of the glass particles were about <53 μm in size and 50% by weight of the glass particles were between about 90-150 μm in size; and “200”—present invention material with 200 mg of bimodal Combeite per mL of bone graft in which fifty percent (50%) by weight of the glass particles were about <53 μm in size and 50% by weight of the glass particles were between about 90-150 μm in size. The exposure was 60 kV for 60 seconds.

### Example 6

### Performance Testing of Bioactive Antibacterial Material

The antibacterial effects of a bimodal particle size distribution of Combeite bioactive glass-ceramic were evaluated. Antibacterial efficacy was investigated against Staphylococcus aureus, Escherichia coli, and Pseudomonas aeruginosa for the glass alone as well as the glass combined with VT Foam Pack Bone Graft Substitute (prepared as described above in Examples 3 and 4 with the addition of a 50 mg/mL group, equivalent to a calcium phosphate: collagen: bimodal glass ratio of approximately 70:20:10).

For testing of the bioactive (BA) glass particles, bimodal glass consisting of 50%<53 μm particles and 50% 90-150 μm particles by weight was added to tryptic soy broth (TSB) at a concentration of 100 mg/mL. The mixture was then inoculated with 0.5×10⁶ colony forming units (CFU)/mL of S. aureus, E. coli, or P. aeruginosa. This concentration represents the physiologic level of bacteria necessary to cause wound infection (Robson, Surg Clin North Am, 1997). After 1, 7, or 14 days, an aliquot was taken, serially diluted, and plated to count colonies. For testing of bimodal bioactive glass with VT Foam Pack Bone Graft Substitute (VT Foam Pack), glass particles were combined with the VT Foam Pack to equivalent concentrations of 50, 100, and 200 mg/mL of bone graft material and kneaded in a sterile manner as per the manufacturer's instructions. The mixture was then inoculated with 0.5×10⁶ CFU/mL and placed in a sealed container. After an incubation period, the mixture was added to broth and shaken vigorously to release any bacteria from the bone graft composite. An aliquot was taken, serially diluted, and plated for counting. Control samples with no added glass were tested in the same manner. Test methods are based on the United States Pharmacopeia <51>Standard.

Table 2 demonstrates the log reductions of the smaller <53 μm particles, which demonstrate efficacy against 3 strains of bacteria at a concentration of 50 mg/mL. Table 3 illustrates the log reductions of the larger 90-150 μm particle size. Negative log reductions indicate that this particle size alone was not effective at reducing levels of bacteria present, even at a higher 100 mg/mL concentration.

Table 4 demonstrates the antibacterial efficacy of a bimodal distribution of particles against 3 strains of bacteria. A greater than 4 log reduction was seen at 1 day, and this antibacterial efficacy was maintained over the course of 14 days indicating that all microorganisms were killed within the first 24 hours of exposure. A 4 log reduction demonstrates 99.99% efficacy.

TABLE 2
<53 μm bioactive glass Log CFU/mL Reductions at 24 hours

| Organism | 50 mg/mL |
|---|---|
| S. aureus | 2.2 |
| P. aeruginosa | >4.6 |
| E. coli | >4.8 |

TABLE 3
90-150 μm bioactive glass Log CFU/mL Reductions at 24 hours

| Organism | 50 mg/mL | 100 mg/mL |
|---|---|---|
| S. aureus | −4.0 | −4.1 |
| P. aeruginosa | −4.2 | −4.1 |
| E. coli | −3.9 | −3.5 |

TABLE 4
100 mg/mL bimodal bioactive glass Log CFU/mL Reductions

| Organism | 1 day | 7 day | 14 day |
|---|---|---|---|
| S. aureus | >4.7 | >4.7 | >4.7 |
| P. aeruginosa | >4.6 | >4.6 | >4.6 |
| E. coli | >4.8 | >4.8 | >4.8 |

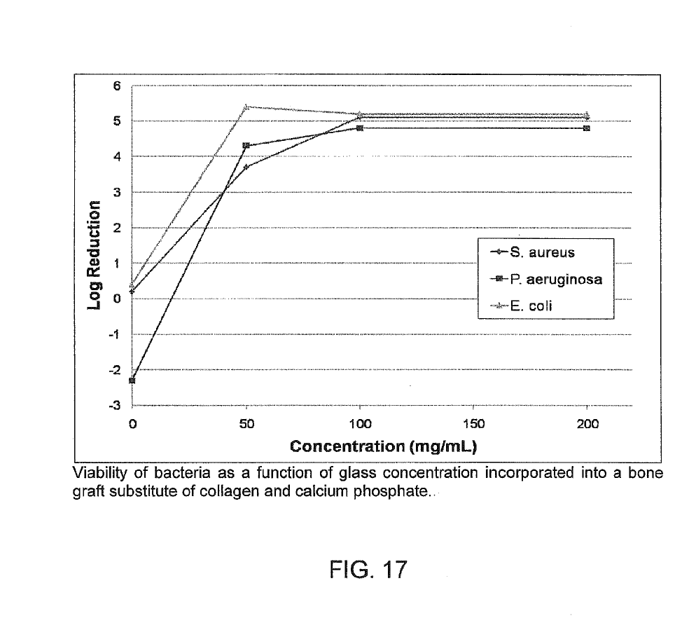

FIG. 17 and Table 5 illustrate the log reduction achieved at 1 day for increasing concentrations of bimodal glass admixed with the VT Foam Pack. Increasing amounts of glass yielded higher log reduction of bacteria until 100 mg/mL was reached. At 100 and 200 mg/mL, BA glass combined with VT Foam Pack yielded an approximate 5 log reduction, or 99.999% efficacy. Without the bioactive glass component there was no antibacterial efficacy observed.

TABLE 5
Vitoss PACK with bimodal glass Log
CFU/mL Reductions at 24 hours

| Organism | 0 mg/mL | 50 mg/mL | 100 mg/mL | 200 mg/mL |
|---|---|---|---|---|
| S. aureus | 0.2 | 3.7 | 5.1 | >5.1 |
| P. aeruginosa | −2.3 | 4.3 | >4.8 | >4.8 |
| E. coli | 0.4 | 5.4 | >5.2 | >5.2 |

Table 6 demonstrates the antibacterial efficacy of VT Foam Pack with 100mg/mL bimodal glass over the course of 28 days. There is a greater than 4 log reduction seen at 1 day, and this antibacterial efficacy was maintained over the course of 28 days indicating that all microorganisms were killed within the first 24 hours of exposure to the antibacterial bone graft.

TABLE 6
100 mg/mL bimodal bioactive glass with
Vitoss Pack Log CFU/mL Reductions

| Organism | 1 day | 7 day | 14 day | 28 day |
|---|---|---|---|---|
| S. aureus | 5.1 | >5.1 | >5.1 | >5.1 |
| P. aeruginosa | >4.8 | >4.8 | >5.1 | >5.1 |
| E. coli | >5.2 | >5.2 | >5.3 | >5.3 |

*Inoculations for all antibacterial efficacy testing were performed at approximately 5.7 log CFU/mL.

This testing demonstrates that a bimodal distribution of BA glass combined with a bone graft substitute of collagen and calcium phosphate possesses antibacterial properties. It is believed that a primary mechanism of action of BA glass is the release of ions into the surrounding medium. Several investigators have found that these ions cause changes in osmotic pressure (Stoor P. Acta Odontol Scand. 1998; 56(3):161-165), an increase in pH (Allan I. Biomaterials. 2001; 22:1683-1687), and release of calcium ions causing membrane perturbations in the bacteria (Munukka E. J Mater Sci: Mater Med. 2008;19:27-32), all factors that play a role in creating inhibitory conditions for bacteria. The small particle sizes in the bimodal distribution have a dissolution rate necessary for immediate ion release, accounting for the demonstrated antibacterial efficacy. Conversely, the larger and slower reacting particles of the bimodal distribution provide benefits for bone healing (Havener M B, Improvements in healing with a bioactive bone graft substitute in a canine metaphyseal defect. Presented at the 55th Annual Meeting of the Orthopedic Research Society, Las Vegas, Nev. (2009)). These dual advantages provide clinical benefits by reducing the incidence of surgical site infections as well as increasing the rate of bone healing.

### Example 7

### Scanning Electron Microscopy (SEM) of Silver-Containing Bioactive Glass

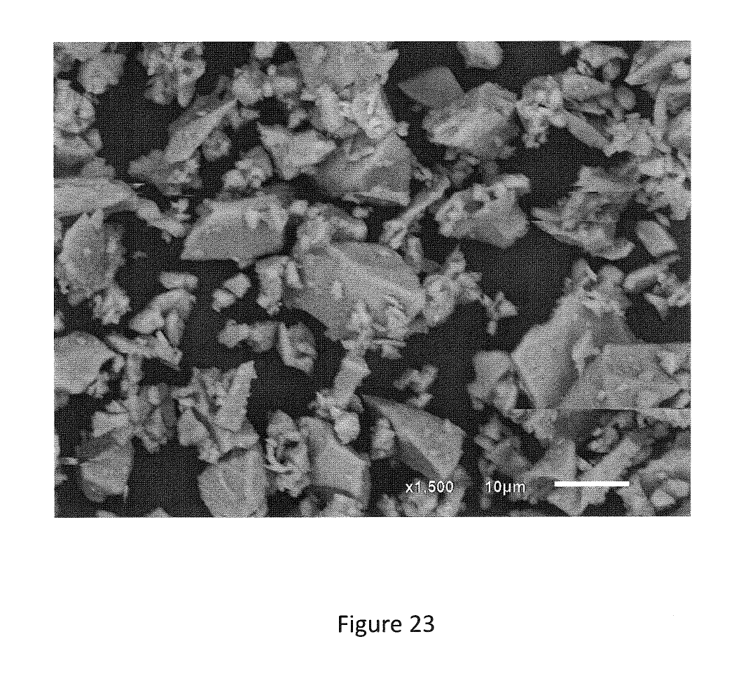

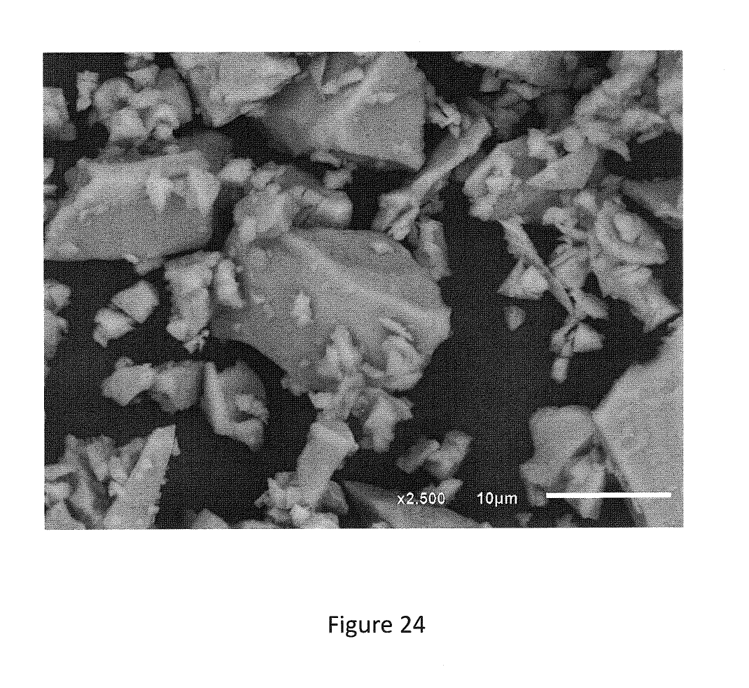

Scanning electron microscopy (SEM) was performed to qualitatively evaluate the silver-containing bioactive glass of the present invention in morsel form. FIGS. 23 and 24 are representative SEM images of silver-containing Combeite glass-ceramic particles <53 μm in size at two different magnifications: 1,500× magnification (FIG. 23) and at 2,500× magnification (FIG. 24).

### Example 8

### Antibacterial Activity of AgBA

The antibacterial effects of silver-containing Combeite bioactive glass-ceramic (AgBA glass ((wt %): CaO, 24.5; P2O5, 6.0; SiO2, 45.0; Na2O, 20.5; Ag2O, 4.0), having a particle size distribution of <53 μm were evaluated. Antibacterial efficacy was investigated against Staphylococcus aureus, Escherichia coli, Bacillus subtilis and Pseudomonas aeruginosa for the glass alone, as well as against S. aureus using the glass combined with VT Foam Pack Bone Graft Substitute.

For testing of the silver-containing AgBA glass particles, silver-containing glass was added to tryptic soy broth (TSB) at concentrations of 3, 5 and 7 mg/mL. The mixture was then inoculated with ˜106 CFU/ml of S. aureus, B. subtilis, E. coli, or P. aeruginosa and incubated at 37 oC. After 1 or 4 days, an aliquot was taken, serially diluted, and plated to count colonies.

For testing of silver-containing AgBA glass with VT Foam Pack Bone Graft Substitute (VT Foam Pack), silver-containing glass particles were combined with the VT Foam Pack to equivalent concentrations of 7, 11, and 15 mg/mL of bone graft material and TSB and kneaded in a sterile manner as per the manufacturer's instructions. The mixture was then inoculated with ˜106 CFU/ml of S. aureus and placed in a sealed container. After the specified incubation period, the mixture was shaken vigorously to release any bacteria from the bone graft composite and an aliquot was taken, serially diluted, and plated for counting. Control samples with TSB alone and a second with PACK with no added glass were tested in the same manner.

Table 7 demonstrates the log reductions of 3 strains of bacteria at a concentration of 6 mg/mL.

TABLE 7
AgBA glass Log CFU/mL Reductions at 4 days

| Organism | 6 mg/mL |
|---|---|
| Control | −1.88 |
| B. subtilis | 5.81 |
| E. coli | 5.67 |
| P. aeruginosa | 4.84 |

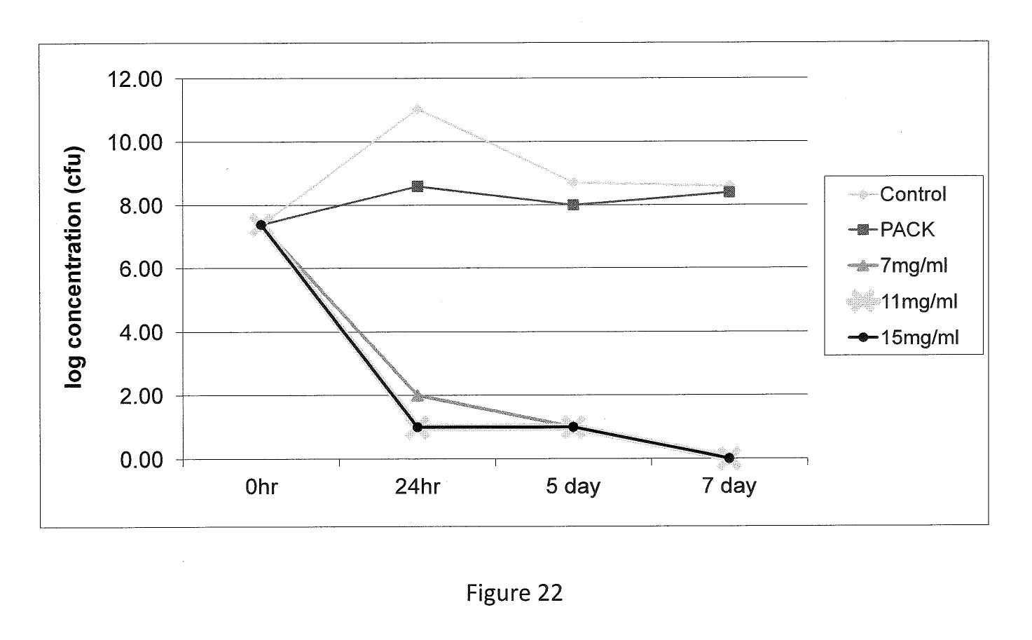

FIG. 22 illustrates the log reduction achieved over 7 days for increasing concentrations of bimodal glass mixed with the VT Foam Pack. Increasing amounts of glass yielded higher log reduction of bacteria. As the graph in FIG. 22 shows, the 11 mg/ml and the 15 mg/ml produced an identical log reduction of bacteria at 24 hours, 5 days and 7 days.

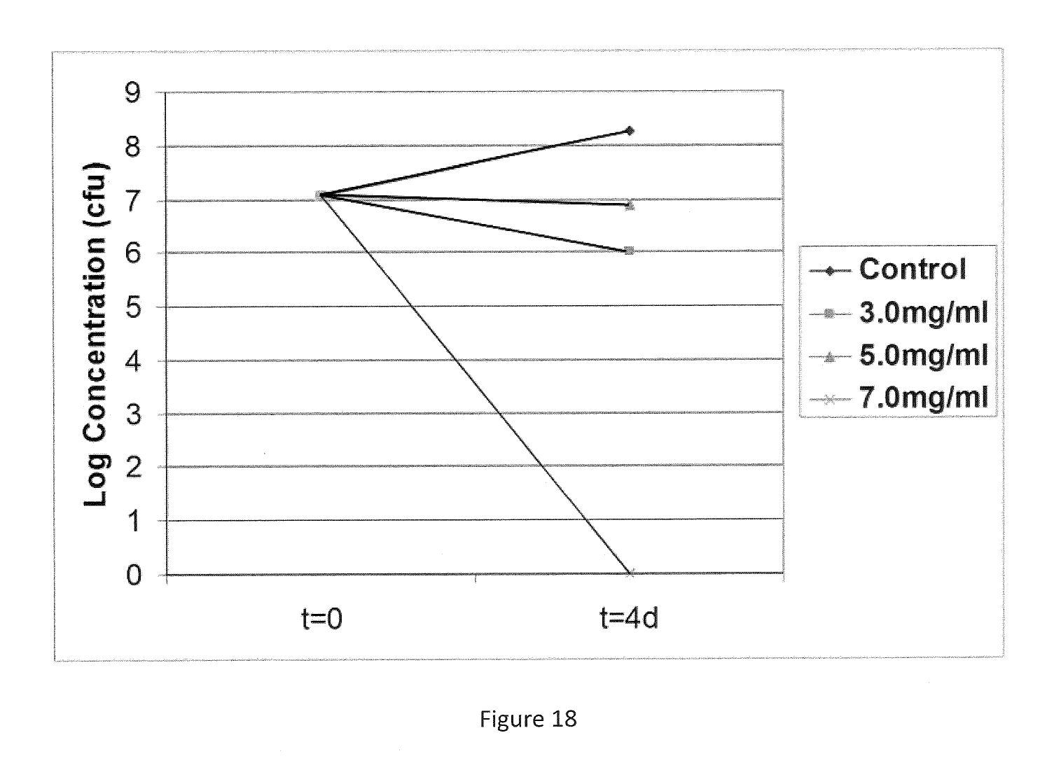

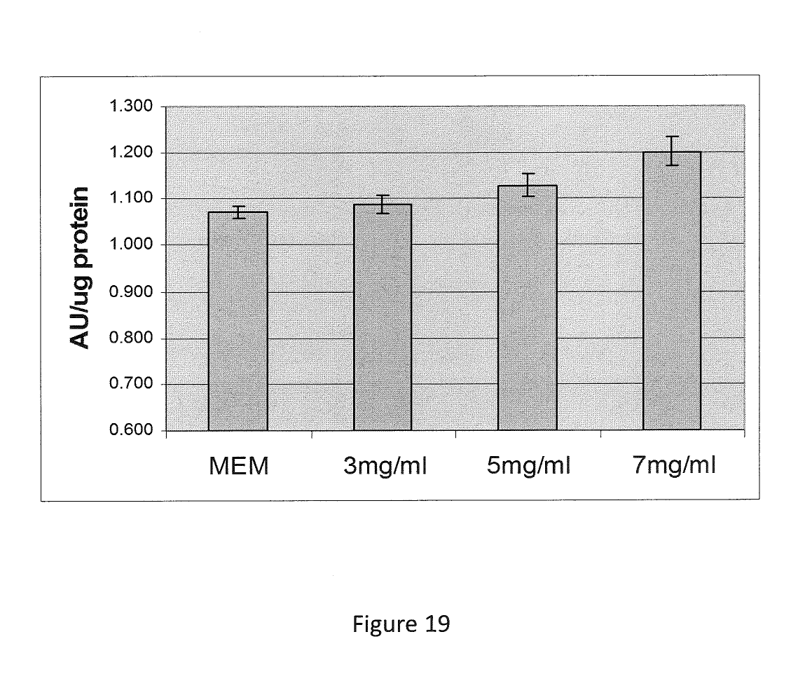

A reduction in bacterial growth was observed at all tested concentrations, with the highest reduction in growth observed at 6 and 7 mg/mL AgBA (FIGS. 18 and Table 7), or 11 and 15 mg/ml of Pack product (FIG. 22). This testing demonstrates that silver BA glass combined with a bone graft substitute of collagen and calcium phosphate possesses antibacterial properties.

### Example 8

### Mammalian Cells Cultured in AgBA Glass-Extracted Media

For cell culture analysis, AgBA glass ((wt %): CaO, 24.5; P2O5, 6.0; SiO2, 45.0; Na2O, 20.5; Ag2O, 4.0), having a particle size distribution of <53μm, was extracted into cell culture media (MEM) at concentrations of 3, 5, or 7 mg/mL for 24 hours at 37 oC in 5% CO2. The media was then applied to cultures of MG-63 osteosarcoma cells, which were incubated at 37 oC over a period of 21 days. At 21 days, alkaline phosphatase (ALP) activity was measured in triplicate. Figure shows the results of an example experiment measuring the level of secreted ALP at increasing concentrations of AgBA. Control samples (MEM with no added glass) were tested in the same manner.

### Example 9

### Osteoblast-Like Cells Growing on AgBA-Doped Scaffolds

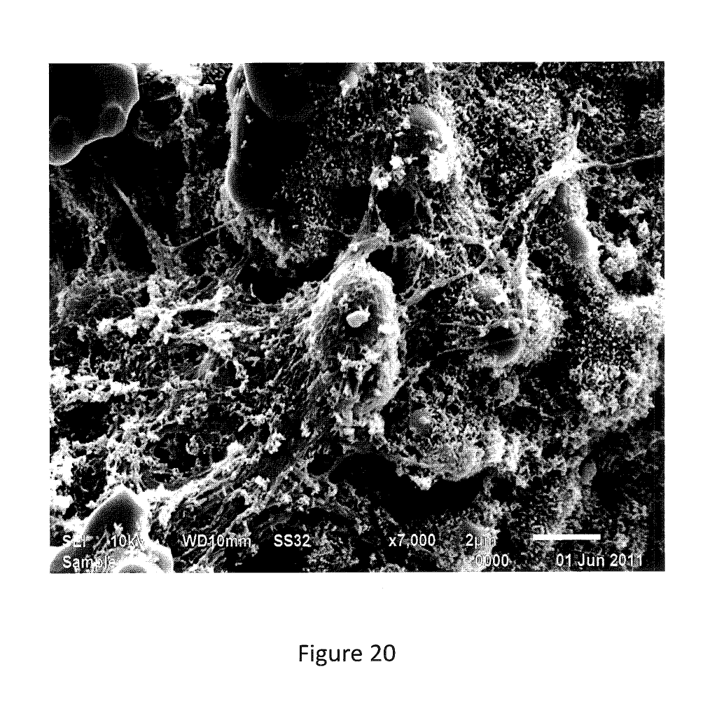

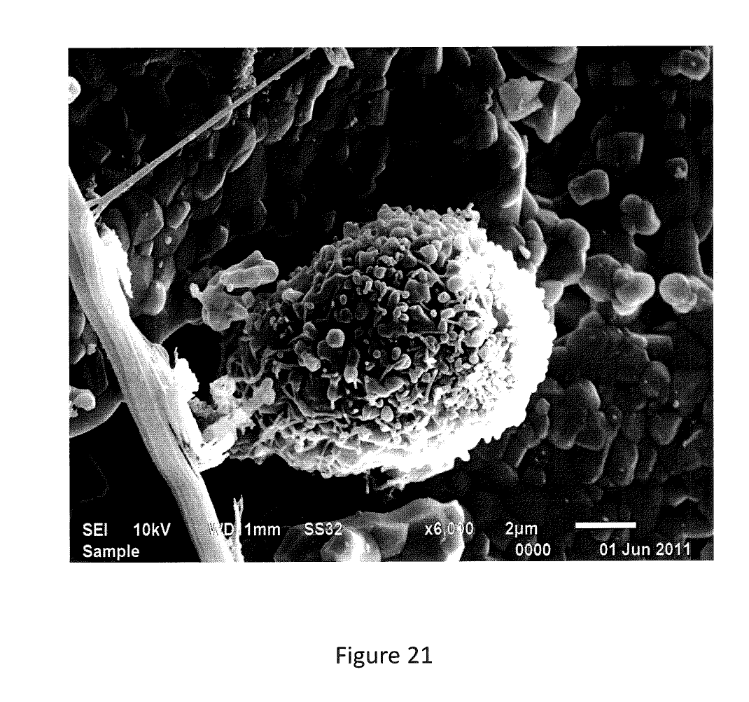

For SEM analysis, osteoblast-like cells were seeded onto Vitoss scaffolds and incubated in AgBA glass extracted culture media at 37 oC with 5% CO2 for 21 days. Media was changed every 2-3 days over the incubation period. At 21 days the scaffold was removed from media, fixed in 2.5% glutaraldehyde in PBS and dehydrated using a graded series of ethanol solutions. The scaffolds were then dried, mounted and sputter coated for SEM imaging. FIGS. 20 and 21 show SEMs of osteoblast-like cells growing on in AgBA doped scaffolds.

Although illustrated and described above with reference to certain specific embodiments and examples, the present invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A bioactive antibacterial composite comprising a biocompatible polymer, a porous calcium phosphate, and a silver-containing bioactive glass.

2. The bioactive antibacterial composite of claim 1, wherein the bioactive glass comprises particles with a bimodal particle size distribution.

3. The bioactive antibacterial composite of claim 1, wherein the biocompatible polymer is collagen.

4. The bioactive antibacterial composite of claim 1, wherein the calcium phosphate is beta-tricalcium phosphate.

5. The bioactive composite of claim 1, wherein the bioactive glass is 45S5 or Combeite.

6. The bioactive composite of claim 1, wherein silver comprises from about 1% to about 15% by weight of the composite composition.

7. The bioactive composite of claim 1, wherein the bioactive glass comprises from about 5% to about 40% by weight of the composite composition.

8. The bioactive antibacterial composite of claim 1, wherein the composite has a total porosity of at least 30% and interconnected macro-, meso- and microporosity.

9. The bioactive antibacterial composite of claim 1, wherein the biocompatible polymer is collagen and the calcium phosphate is beta-tricalcium phosphate.

10. The bioactive antibacterial composite of claim 9, wherein the biocompatible polymer comprises from about 10% to about 20% by weight of the composite, the beta-tricalcium phosphate comprises from about 50% to about 75% by weight of the composite and the bioactive glass comprises from about 10% to about 35% by weight of the composite.

11. The bioactive antibacterial composition of claim 2, wherein the bioactive glass comprises particles with a particle size of less than about 53 microns (μm) and particles of a particle size range from about 90 μm to about 150 μm.

12. The bioactive antibacterial composition of claim 2, wherein the bioactive glass comprises particles with a particle size range of about 32 μm to about 90 μm and particles of a particle size range from about 90 μm to about 150 μm.

13. The bioactive antibacterial composite of claim 1, wherein the biocompatible polymer comprises from about 10% to about 20% by weight of the composite, the porous calcium phosphate comprises from about 50% to about 75% by weight of the composite, silver comprises from about 1% to about 10% by weight of the composite, and the bioactive glass comprises from about 10% to about 35% by weight of the composite.

14. A bioactive antibacterial composite comprising a biocompatible polymer, a porous calcium phosphate, and silver-containing bioactive glass, wherein about 50% by weight of the bioactive glass comprises particles with a particle size of less than about 53 μm and about 50% by weight of the bioactive glass comprises particles having a particle size range from about 90 μm to about 150 μm.

15. The bioactive antibacterial composite of claim 14, wherein silver comprises from about 1% to about 15% by weight of the composite composition.

16. A bioactive antibacterial composite comprising a biocompatible polymer, a porous calcium phosphate, and silver-containing bioactive glass, wherein about 50% by weight of the bioactive glass comprises particles with a particle size range from about 32 μm to about 90 μm and about 50% by weight of the bioactive glass comprises particles having a particle size range from about 90 μm to about 150 μm.

17. The bioactive antibacterial composite of claim 16, wherein silver comprises from about 1% to about 15% by weight of the composite composition.

18. A bioactive antibacterial material comprising silver-containing bioactive glass, wherein the bioactive glass comprises particles with a particle size of less than about 53 μm and particles of a particle size range from about 90 μm to about 150 μm.

19. The bioactive antibacterial composite of claim 18, wherein silver comprises from about 1% to about 15% by weight of the composite composition.

20. The bioactive antibacterial material of claim 18, wherein about 50% by weight of the material has bioactive glass particles having a particle size of less than about 53 μm and about 50% by weight of the material has bioactive glass particles having a particle size range from about 90 μm to about 150 μm.

21. A bioactive antibacterial material comprising silver-containing bioactive glass, wherein the bioactive glass comprises particles with a particle size range from about 32 μm to about 90 μm and particles of a particle size range from about 90 μm to about 150 μm.

22. The bioactive antibacterial composite of claim 21, wherein silver comprises from about 1% to about 15% by weight of the composite composition.

23. The bioactive antibacterial material of claim 21, wherein about 50% by weight of the material has bioactive glass particles having a particle size range from about 32 μm to about 90 μm and about 50% by weight of the material has bioactive glass particles having a particle size range from about 90 μm to about 150 μm.

24. A bioactive antibacterial composite comprising a biocompatible polymer, a porous calcium phosphate, and a silver-containing bioactive glass, wherein the bioactive glass is comprised of particles with a particle size of less than about 150 μm.

25. The bioactive antibacterial composite of claim 24, wherein silver comprises from about 1% to about 15% by weight of the composite composition.

26. The bioactive antibacterial composition of claim 24, wherein the bioactive glass is comprised of particles having a bimodal particle size distribution.

27. A method for repairing a defect in bone and preventing surgical site infection comprising the step of administering to the bone an implant comprising silver-containing bioactive glass.

28. The method of claim 27, wherein the bioactive glass includes bimodal particles with a particle size from about less than 53 μm and particles with a particle size range of from about 90 μm to about 150 μm.

29. The method of claim 27, wherein the bioactive glass comprises particles with a particle size range from about 32 μm to about 90 μm and particles of a particle size range from about 90 μm to about 150 μm.

30. The method of claim 27, wherein silver comprises from about 1% to about 15% by weight of the implant.

31. The method of claim 27, wherein the defect is a defect in the spine.

32. The method of claim 27, wherein the defect is a defect in the vertebral body.

33. A method for repairing a damaged bone or tooth comprising placing in the bone or jaw an implant comprising a biocompatible polymer, calcium phosphate and silver-containing bioactive glass.

34. The method of claim 33, wherein silver comprises from about 1% to about 15% by weight of the implant.

35. The method of claim 33, wherein the bioactive glass includes particles with a particle size from about less than 53 μm and particles with a particle size range of from about 90 μm to about 150 μm.

36. The method of claim 33, wherein the bioactive glass comprises particles with a particle size range from about 32 μm to about 90 μm and particles of a particle size range from about 90 μm to about 150 μm.
